(19) United States
(12) Patent Application Publication
(10) Pub. No.: US 2004/0265393 A1
(43) Pub. Date: Dec. 30, 2004

(54) Non-invasive intravascular thrombolysis using modified ultrasound techniques

(76) Inventors: Evan C. Unger, Tucson, AZ (US); Reena Zutshi, Tucson, AZ (US); Terry O. Matsunaga, Tucson, AZ (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/865,972

(22) Filed: Jun. 10, 2004

Related U.S. Application Data
(60) Provisional application No. 60/477,927, filed on Jun. 13, 2003.

(51) Int. Cl. A61K 33/00; A61N 1/30

(52) U.S. Cl. 424/600; 604/20

(57) ABSTRACT

A non-invasive method for disrupting a blood clot within the vasculature of a patient using new ultrasound techniques is provided. Lipid vesicles containing a gas or gaseous precursor are administered intravascularly to the patient and ultrasound having a power greater than about 0.5 Watts/cm² to about 20 Watts/cm² for about 10% to about 80% of the duty cycle is applied to the patient for a period of time sufficient to induce rupture of the vesicles adjacent to the site of the blood clot, thereby disrupting the blood clot. Administration of thrombolytic biological agents is not required. Optionally, progress of clot disruption can be monitored using magnetic resonance imaging.

This application claims the benefit under 35 U.S.C. § 119(e) of U.S. Provisional Application No. 60/477,927, filed Jun. 13, 2003, the entire contents of which is incorporated herein by reference.

### BACKGROUND OF THE INVENTION

1. Field of the Invention

This invention relates generally to the field of therapeutic ultrasound, and more specifically, to the use of stabilized gas-filled vesicles for sonolysis of a vascular blood clot, optionally monitored by magnetic resonance imaging (MRI).

2. Background

There are a variety of imaging techniques that have been used to diagnose disease in humans. One of the first imaging techniques employed was X-rays. In X-rays, the images produced of the patients' body reflect the different densities of body structures. To improve the diagnostic utility of this imaging technique, contrast agents are employed to increase the density of tissues of interest as compared to surrounding tissues to make the tissues of interest more visible on X-ray. Barium and iodinated contrast media, for example, are used extensively for X-ray gastrointestinal studies to visualize the esophagus, stomach, intestines and rectum. Likewise, these contrast agents are used for X-ray computed tomographic studies (that is, computer assisted tomography or CAT) to improve visualization of the gastrointestinal tract and to provide, for example, a contrast between the tract and the structures adjacent to it, such as the vessels or lymph nodes. Such contrast agents permit one to increase the density inside the esophagus, stomach, intestines and rectum to allow differentiation of the gastrointestinal system from surrounding structures.

Magnetic resonance imaging (MRI) is a relatively new imaging technique that, unlike X-rays, does not utilize ionizing radiation. Like computer-assisted tomography (CAT), MRI can make cross-sectional images of the body; however, MRI has the additional advantage of being able to make images in any scan plane (i.e., axial, coronal, sagittal or orthogonal). Unfortunately, the full utility of MRI as a diagnostic modality for the body is hampered by the need for new or better contrast agents. Without suitable agents, it is often difficult to use MRI to differentiate the target tissue from adjacent tissues. If better contrast agents were available, the overall usefulness of MRI as an imaging tool would improve, and the diagnostic accuracy of this modality would be greatly enhanced.

MRI employs a magnetic field, radio frequency energy and magnetic field gradients to make images of the body. The contrast or signal intensity differences between tissues mainly reflect the T1 (longitudinal) and T2 (transverse) relaxation values and the proton density (effectively, the free water content) of the tissues. In changing the signal intensity in a region of a patient by the use of a contrast medium, several possible approaches are available. For example, a contrast medium could be designed to change the T1, the T2 or the proton density.

In the past, attention has mainly been focused on paramagnetic contrast media for MRI. Paramagnetic contrast agents contain unpaired electrons, which act as small local magnets within the main magnetic field to increase the rate of longitudinal (T1) and transverse (T2) relaxation. Most paramagnetic contrast agents are metal ions, which in most cases are toxic. In order to decrease toxicity, these metal ions are generally chelated using ligands. The resultant paramagnetic metal ion complexes have decreased toxicity. Metal oxides, most notably iron oxides, have also been tested as MRI contrast agents. While small particles of iron oxide, e.g., under 20 nm diameter, may have paramagnetic relaxation properties, their predominant effect is through bulk susceptibility. Therefore magnetic particles have their predominant effect on T2 relaxation. Nitroxides are another class of MRI contrast agent that is also paramagnetic. These have relatively low relaxivity and are generally less effective than paramagnetic ions as MRI contrast agents. All of these contrast agents can suffer from some toxic effects in certain contexts of use and none of them are ideal for use as perfusion contrast agents by themselves.

Certain existing MRI contrast agents suffer from a number of limitations. For example, positive contrast agents are known to exhibit increased image noise arising from intrinsic peristaltic motions and motions from respiration or cardiovascular action. Positive contrast agents, such as Gd-DTPA, are subject to the further complication that the signal intensity depends upon the concentration of the agent as well as the pulse sequence used. Absorption of contrast agent from the gastrointestinal tract, for example, complicates interpretation of the images, particularly in the distal portion of the small intestine, unless sufficiently high concentrations of the paramagnetic species are used (Kornmesser et al., Magn. Reson. Imaging 6:124 (1988)). Negative contrast agents, by comparison, are less sensitive to variation in pulse sequence and provide more consistent contrast, but typically exhibit superior contrast to fat. However on T1-weighted images, positive contrast agents exhibit superior contrast versus normal tissue. Since most pathological tissues exhibit longer T1 and T2 than normal tissue, they will appear dark on T1-weighted and bright on T2-weighted images. This would indicate that an ideal contrast agent should appear bright on T1-weighted images and dark on T2-weighted images. Many of the currently available MRI contrast media fail to meet these dual criteria.

Toxicity is another problem with certain existing contrast agents. With any drug there is some toxicity, the toxicity generally being dose related. With the ferrites there are often symptoms of nausea after oral administration, as well as flatulence and a transient rise in serum iron. The paramagnetic contrast agent Gd-DTPA is an organometallic complex of gadolinium coupled with the complexing agent diethylene triamine pentaacetic acid. Without coupling, the free gadolinium ion is highly toxic. Furthermore, the peculiarities of the gastrointestinal tract, for example, wherein the stomach secretes acids and the intestines release alkalines, raise the possibility of decoupling and separation of the free gadolinium or other paramagnetic agent from the complex as a result of pH changes during gastrointestinal use. Certainly, minimizing the dose of paramagnetic agents is important for minimizing any potential toxic effects.

In the work on MRI contrast agents described in U.S. application Ser. No. 07/507,125, filed Apr. 10, 1990, gas is used in combination with polymer compositions and paramagnetic or superparamagnetic agents as MRI contrast agents. The gas stabilized by the polymers function as an effective susceptibility contrast agent to decrease signal intensity on T2 weighted images and that such systems are particularly effective for use as gastrointestinal MRI contrast media.

Widder et al. published application EP-A-0 324 938 discloses stabilized microbubble-type ultrasonic imaging agents produced from heat-denaturable biocompatible protein, e.g., albumin, hemoglobin, and collagen.

There is also mentioned a presentation believed to have been made by Moseley et al., at a 1991 Napa, Calif. meeting of the Society for Magnetic Resonance in Medicine, which is summarized in an abstract entitled “Microbubbles: A Novel MR Susceptibility Contrast Agent.” The microbubbles that are utilized comprise air coated with a shell of human albumin.

For intravascular use, however, it is advantageous that any gas bubbles be stabilized with flexible non-protein compounds to avoid bubble shells that are often brittle and inflexible because a brittle coating limits the capability of the bubble to expand and collapse as the bubble encounters different pressure regions within the body (e.g., moving from the venous system into the arteries upon circulation through the heart). A brittle shell may break and lose the gas, thereby limiting the effective period of time during which useful contrast can be obtained in vivo from these microbubble contrast agents. Also, such brittle, broken fragments can be potentially toxic.

Quay published application WO 93/05819 discloses that gases with high diffusibility factors (i.e., Q numbers) are ideal stable gases. For example, sorbitol is used to increase viscosity, which in turn extends the life of a microbubble in solution.

Lanza et al. published application WO 93/20802 discloses acoustically reflective oligolamellar liposomes, with increased aqueous space between bilayers in which smaller liposomes can be nested within bilayers in a nonconcentric fashion to internally separate bilayers. Use of such liposomes as ultrasonic contrast agents to enhance ultrasonic imaging, and to monitor a drug delivered therein to a patient, is also described.

D'Arrigo U.S. Pat. Nos. 4,684,479 and 5,215,680 disclose, respectively, gas-in-liquid emulsions and lipid-coated microbubbles.

Despite technical improvements to the ultrasound modality, the images obtained are still subject to further refinement, particularly in regards to imaging of the vasculature and tissues that are perfused with a vascular blood supply. Toward that end, contrast agents are typically used to aid in the visualization of the vasculature and vascular-related organs. In particular, microbubbles or vesicles are desirable as contrast agents for ultrasound because the reflection of sound at an interface created at the surface of a vesicle is extremely efficient. These vesicles are also useful in therapeutic methods in conjunction with ultrasound such as for performing surgery in the vasculature (U.S. Pat. No. 6,576,220) or effecting treatment by delivering drugs or nucleic acid materials for localized therapy (U.S. Pat. Nos. 6,443,898 and 5,770,222). It is known to produce suitable contrast agents comprising microbubbles by first placing an aqueous suspension or powder (i.e., a bubble coating agent), preferably comprising lipids or albumin, into a vial or container (e.g. U.S. Pat. No. 6,551,576). A gas phase is then introduced above the aqueous suspension or powder phase in the remaining portion, or headspace, of the vial. The vial is then shaken prior to use in order to form the microbubbles. It will be appreciated that, prior to shaking, the vial contains an aqueous suspension or solid phase and a gaseous phase. A wide variety of bubble or vesicle coating agents may be employed in the aqueous suspension phase or dry powder solid phase, such as those comprised of lipids (e.g. Definity®, sold by Bristol Meyers Squibb Medical Imaging or Imagent®, developed by Alliance Pharmaceutical), those comprising proteins such as albumin (e.g. Optison® sold by Amersham), albumin and dextrose (PESDA, U.S. Pat. No. 5,648,098) or polymers (U.S. Pat. No. 5,512,268). Likewise, a wide variety of different gases may be employed in the gaseous phase. In particular, however, fluorinated gases, such as sulfur hexafluoride or perfluorocarbon gases such as perfluoropropane (perflutren) may be used. See, for example, Unger et al., U.S. Pat. No. 5,769,080. Mixtures of gases are also used, such as perfluorohexane and nitrogen in Imagent®. The disclosure of each of the above-described patents is hereby incorporated in by reference in its entirety.

In accordance with the present invention it has been discovered that stabilized gas-filled vesicles are extremely effective, non-toxic contrast agents for noninvasive ultrasound lysis of a blood clot, optionally simultaneously monitored with MRI.

### A BRIEF DESCRIPTION OF THE FIGURE

FIG. 1 is a graph showing the effects of the invention methods on the dissolution rate of fluorescein labeled fibrinogen human blood clots (n=6).

### SUMMARY OF THE INVENTION

The present invention is based on the discovery of modified ultrasound parameters that allow non-invasive ultrasound applied to rupture intravascularly administered gas-filled vesicles to disrupt a blood clot within the peripheral vasculature of a patient without damage to the surrounding vasculature or substantial discomfort to the patient.

Accordingly, the present invention provides methods for disrupting a blood clot within the peripheral vasculature of a patient by (a) administering intravascularly to the patient an aqueous formulation of vesicles comprising a gas or gaseous precursor, and a lipid-stabilizing compound. Ultrasound having a power of about 0.1 Watts/cm² to about 30 Watts/cm² with a mechanical index less than or equal to 3.0 for about 10% to about 80% of the duty cycle is applied to the patient at the site of the blood clot for a period of time sufficient to induce rupture of the vesicles adjacent to the site of the blood clot, thereby disrupting the blood clot.

### DETAILED DESCRIPTION OF THE INVENTION

In the invention methods, the patient is administered the gas-filled vesicles intravascularly, the vesicles pass to a point adjacent to the blood clot, and ultrasonic energy directed to the region of the patient having the blood clot is used to rupture the vesicles, thereby carrying out thrombolysis. Optionally, an imaging modality, such as magnetic resonance imaging (MRI), can simultaneously be used to monitor passage of the gas-filled vesicles to the intravascular site of the blood clot for rupture. A second MRI scanning to determine the success of the ultrasonic thrombolysis can follow the application of ultrasound. The scanning and application of ultrasound can be performed repeatedly until the desired effect is achieved.

The vesicles used in the invention methods comprise a gas or gas precursor, such as a perfluorocarbon having no more than 10 carbon atoms, and serve to enhance thrombolysis upon rupture of the vesicles by ultrasonic energy as well as being an excellent contrast medium for monitoring the process using MRI. The gas-filled vesicles are stabilized by comprising a biocompatible lipid, and may optionally further comprises a therapeutic agent that is released to a localized region of a patient upon rupture of the vesicles by ultrasound. For example the therapeutic agent can be a thrombolytic, such as tissue plasminogen activator (tPA), either natural or recombinant, urokinase, pro-urokinase, reteplase, wafarins, tenecteplase, streptokinase, hirudin, or an anticoagulant such as heparin, e.g. heparin sulfate and low molecular weight heparin or nitrous oxide. Additional therapeutic agents that can advantageously be delivered by the vesicles according to the invention methods are disclosed in U.S. Pat. Nos. 5,770,222 and 6,443,898, each of which is incorporated hereby by reference in its entirety.

Provided that the circulation half-life of the vesicles is sufficiently long, the vesicles will generally pass through the target vasculature as they pass through the body. By focusing the rupture inducing sound waves on the selected tissue to be treated, the vesicles will be ruptured locally in the target vasculature. As a further aid to targeting, antibodies, carbohydrates, peptides, glycopeptides, glycolipids, lectins, glycoconjugates, and synthetic and natural polymers, such as and not limited to polyethylene glycol, polyvinylpyrrolidone, polyvinylalcohol, which may be incorporated onto the surface via alkylation, acylation, sterol groups or derivatized head groups of phospholipids such as dioleoylphosphatidylethanolamine (DOPE), dipalmitoylphosphatidylethanolamine (DPPE), or disteroylphosphatidylethanolamine (DSPE), may also be incorporated into the surface of the vesicles.

The present invention can be carried out, often with considerable attendant advantage, by using gaseous precursors to form the gas of the gas-filled vesicles. These gaseous precursors can be activated by a number of factors, but preferably are temperature activated. Such a gaseous precursor is a compound that, at a selected activation or transition temperature, changes phases from a liquid or solid to a gas. Activation thus takes place by increasing the temperature of the compound from a point below, to a point above, the activation or transition temperature. The lipid used in formation of the vesicles can be in the form of a monolayer or bilayer, and the mono- or bilayer lipids can be used to form a series of concentric mono- or bilayers. Thus, the lipid can be used to form a unilamellar liposome (comprised of one monolayer or bilayer lipid), an oligolamellar liposome (comprised of two or three monolayer or bilayer lipids) or a multilamellar liposome (comprised of more than three monolayer or bilayer lipids). The biocompatible lipid can be a combination comprising a phospholipid. Optionally, if the vesicles used in the invention methods are to serve as a contrast medium, a paramagnetic or superparamagnetic compound can also be encapsulated by or attached to the vesicles.

These and other aspects of the invention will become more apparent from the following detailed description, which contains numerous details in order to provide a thorough understanding of the disclosed embodiments of the invention. However, it will be apparent to those skilled in the art that the embodiments can be practiced without these specific details. In other instances, devices, methods, procedures, and individual components that are well known in the art have not been described in detail herein.

Definitions:

As used herein, “a” or “an” can mean one or more than one of an item.

A “gaseous precursor,” as used herein, is a liquid or a solid at the temperature of manufacture and storage, but becomes a gas at least at or during the time of use.

As used herein, the term “simultaneous” means that ultrasound and magnetic resonance imaging can be applied concurrently or synchronously; sequentially or successively; such that visualization of the passage of the vesicles to the site of the blood clot as well as disruption of vesicles and tissues by ultrasound is observed. Thus, ultrasound and magnetic resonance can be performed at the same time, or one can be followed by the other. The use of magnetic resonance imaging together with ultrasound improves the accuracy of currently available imaging modalities by precisely confirming the location of the vesicles because the entire body can be scanned by magnetic resonance imaging. Once located in the region of the body where lysis of a blood clot is desired, the vesicles can be ruptured by ultrasound, adding destructive energy to lyse the blood clot.

As used herein, the term “a heparin” includes low molecular weight heparin derivatives as well as unfractionated heparin that have anti-coagulant activity. Generally, heparins have a molecular weight in the range from about 3,000 to about 40,000. Heparin consists of sulfated single chain glycoaminoglycans of variable length. Low molecular weight heparins are a group of derivatives of unfractionated heparin whose molecular weights have been well characterized by E. A. Johnson et al., Carbohydr Res 51:119-27, 1976, which is incorporated herein by reference in its entirety. Although widely used in Europe, the only low molecular weight heparins currently available in the United States are enoxaprin™ (Lovenox, Rhone-Poulenc Rorer) and fragmin® (Pfizer). Heparin is highly lipophilic, non-toxic, and is known to bind with affinity to oxidized-LDL-cholesterol. This fact has been utilized for many years in the approach to drug resistant hypercholesterolemia of heparin induced LDL precipitation. As a result of these studies, intravenous dosing of heparin is well known by those of skill in the art.

As used herein the term “thrombolytic agent” includes drugs that interfere with the body's ability to form blood clots (or the clot-promoting effects of platelets). Among such drugs are “tissue plasminogen activator (tPA)”, which refers to an enzyme that occurs naturally in man and causes blood clots to dissolve, as well as to a man-made protein manufactured by recombinant DNA technology. Recombinantly produced tPA is known generically as “Alteplase” and has various commercial designations. Additional “thrombolytic agents” include, for example, warfarin (Coumadin®), aspirin, and nonsteroidal anti-inflammatory drugs (NSAIDs), such as ibuprofen (Motrin®), naproxen (Naprosyn®), and nabumetone (Relafen®). Specific platelet inhibitors, for example, clopidogrel (Plavix®), do not appear to interact with alteplase and increase the risk of bleeding. Those of skill in the art will know how to distinguish which of these thrombolytic agents are intended for delivery intravenously, which are intended for delivery intraarterially, and which can be administered either intravenously or intraarterially for treatment of a blood clot. Such drugs can be injected either at the treatment site or at a distal site.

In addition to thrombolytic agents, certain other drugs or therapeutic agents may advantageously be delivered using the invention methods. For example, antihyperlipidemic agents, such as the statins and high density lipids (HDLs), can be co-administered at the time of thrombolytic treatment.

As used herein, the term “mechanical index” (MI) is defined as follows: MI=Pa/√Fc where Pa=acoustic pressure in Mpa and √Fc=square root of center frequency. MI is the counterpart of the international term “cavitation index” (CI). These indices are measures of the potential for mechanical damage to tissue exposed to intense pulses of ultrasound. These indices are based on the peak rarefactional pressure and on the frequency of the ultrasound pulse.

As used herein, the term “duty cycle” is defined by the following:

Duty cycle=pulse duration (on time)/pulse period (on and off time).

“Ultrasound imaging” is performed on the tissues of interest and ultrasound energy can be used to activate or rupture the vesicles once they reach their intended tissue destination. Focused or directed ultrasound, as distinguished from non-focused ultrasound, refers to the application of ultrasound energy to a particular region of the body, such that the ultrasound energy is concentrated to a selected area or target zone. In addition, “directed” refers to the magnetic resonance which guides the ultrasound by visualizing the vesicles and the target zone; and simultaneous with ultrasound, visualizing the disruption of tissues thereby. “Noninvasive” refers to the disruption or disturbance of internal body tissues without an incision in the skin.

Ultrasound, as defined in accordance with the present invention, refers to lysis or disruption of a blood clot or thrombus in the vasculature; and the activation or rupture of vesicles adjacent to vascular tissue by ultrasonic energy. Ultrasound is a diagnostic imaging technique that is unlike nuclear medicine and X-rays since it does not expose the patient to the harmful effects of ionizing radiation. Moreover, unlike magnetic resonance imaging, ultrasound is relatively inexpensive and can be conducted as a portable examination. In using the ultrasound technique, sound is transmitted into a patient or animal via a transducer. When the sound waves propagate through the body, they encounter interfaces from tissues and fluids. Depending on the acoustic properties of the tissues and fluids in the body, the ultrasound sound waves are partially or wholly reflected or absorbed. When sound waves are reflected by an interface they are detected by the receiver in the transducer and processed to form an image. The acoustic properties of the tissues and fluids within the body determine the contrast that appears in the resultant image. Alternatively, ultrasound can be used to visualize the vesicles and magnetic resonance imaging can be used to activate the vesicles. In addition, the strength of ultrasound energy can be at an intensity to result in rupture or activation of vesicles. The activation of the vesicles in turn disrupts the adjacent tissue such that necrosis of the tissue results.

Any of the various types of diagnostic ultrasound imaging devices can be employed in the practice of the invention, the particular type or model of the device not being critical to the method of the invention. Also suitable are devices designed for administering ultrasonic hyperthermia, such devices being described in U.S. Pat. Nos. 4,620,546, 4,658,828, and 4,5.86,512, the disclosures of each of which are hereby incorporated herein by reference in their entirety. Preferably, the device employs a resonant frequency (RF) spectral analyzer. The transducer probes can be applied externally or can be implanted. Ultrasound is generally initiated at lower intensity and duration, preferably at peak resonant frequency, and then intensity, time, and resonant frequency increased until the microsphere ruptures. More specifically, in the practice of the invention methods

“Vesicle” refers to a spherical entity that is characterized by the presence of an internal void. Preferred vesicles are formulated from lipids, including the various lipids described herein. In any given vesicle, the lipids can be in the form of a monolayer or bilayer, and the mono- or bilayer lipids can be used to form one or more mono- or bilayers. In the case of more than one mono- or bilayer, the mono- or bilayers are generally concentric. The vesicles described herein include such entities commonly referred to as liposomes, micelles, bubbles, microbubbles, aerogels, clathrate bound vesicles, and the like. Thus, the lipids can be used to form a unilamellar vesicle (comprised of one monolayer or bilayer), an oligolamellar vesicle (comprised of about two or about three monolayers or bilayers), or a multilamellar vesicle (comprised of more than about three monolayers or bilayers). The internal void of the vesicles can be filled with a liquid, including, for example, an aqueous liquid, a gas, a gaseous precursor, and a solid or solute material, including, for example, a targeting ligand and a bioactive agent, as desired.

“Liposome” refers to a generally spherical cluster or aggregate of amphipathic compounds, including lipid compounds, typically in the form of one or more concentric layers. Most preferably the gas-filled liposome is constructed of a single layer (i.e. unilamellar) or a single monolayer of lipid. A wide variety of lipids can be used to fabricate the liposomes including phospholipids and non-ionic surfactants (e.g. niosomes). Most preferably the lipids comprising the gas-filled liposomes are in the gel state at physiological temperature. The liposomes can be cross-linked or polymerized and can bear polymers such as polyethylene glycol on their surfaces.

Targeting ligands directed to blood clots can be bound to the surface of the gas-filled liposomes. A targeting ligand is a substance that is bound to a vesicle and directs the vesicle to a particular cell type or molecule, such as platelets or fibrin. For example, 7E3 is an IgG1 monoclonal antibody that binds to the complexed glycoprotein IIb/IIIa contained in platelets. T2G1s monoclonal antifibrin antibody fragment (Fab′)binds to arterial thrombi. The targeting ligand can be bound to the vesicle by covalent or non-covalent bonds. The liposomes may also be referred to herein as lipid vesicles. Most preferably the liposomes are substantially devoid of water in their interiors.

“Micelle” refers to colloidal entities that form from lipidic compounds when the concentration of the lipidic compounds, such as lauryl sulfate, is above a critical concentration. Since many of the compounds that form micelles also have surfactant properties (i.e. ability to lower surface tension and both water and fat loving-hydrophilic and lipophilic domains), these same materials may also be used to stabilize bubbles. In general these micellar materials prefer to adopt a monolayer or hexagonal H₂ phase configuration, yet may also adopt a bilayer configuration. When a micellar material is used to form a gas-filled vesicle, the compounds will generally adopt a radial configuration with the aliphatic (fat loving) moieties oriented toward the vesicle and the hydrophilic domains oriented away from the vesicle surface. For targeting to endothelial cells, the targeting ligands can be attached to the micellar compounds or to amphipathic materials admixed with the micellar compounds. Alternatively, targeting ligands can be adsorbed to the surface of the micellar materials stabilizing the vesicles.

“Aerogel” refers to structures that are similar to vesicles, except that the internal structure of the aerogels is generally comprised of multiple small voids rather than one void. Additionally the aerogels are preferably constructed of synthetic materials (e.g. a foam prepared from baking resorcinol and formaldehyde), however natural materials such as polysaccharides or proteins may also be used to prepare aerogels. Targeting ligands can be attached to the surface of the aerogel.

“Clathrates” are generally solid materials that bind the vesicles as a host rather than coating the surface of the vesicle. A solid, semi-porous, or porous clathrate may serve as the agent stabilizing the vesicle; however, the clathrate itself does not coat the entire surface of the vesicle. Rather, the clathrate forms a structure known as a “cage” having spaces into which the vesicles may fit. One or more vesicles can be adsorbed by the clathrate. Similar to vesicles, one or more surfactants can be incorporated with the clathrate and these surfactants will help to stabilize the vesicle. The surfactants will generally coat the vesicle and help to maintain the association of the vesicle with the clathrate. Useful clathrate materials for stabilizing vesicles include porous apatites, such as calcium hydroxyapatite, and precipitates of polymers with metal ions, such as alginic acid with calcium salts. Targeting ligands directed to endothelial cells can be incorporated into the clathrate itself or into the surfactant material used in association with the clathrate.

“Magnetic resonance imaging” (MRI) uses a static main magnetic field; pulsed radiofrequency energy and pulsed magnetic gradients to create images, i.e. to visualize the vesicles. The radiofrequency and electrical gradients can be used to cause local energy deposition and activate the vesicles; however, ultrasound is the preferred energy for the purpose of activating the vesicles. In carrying out the magnetic resonance imaging method of the present invention, the contrast medium can be used alone, or in combination with other diagnostic, therapeutic or other agents. Such other agents include excipients such as flavoring or coloring materials. The magnetic resonance imaging techniques which are employed are conventional and are described, for example, in D. M. Kean and M. A. Smith, Magnetic Resonance Imaging: Principles and Applications, (William and Wilkins, Baltimore 1986). Contemplated MRI techniques include, but are not limited to, nuclear magnetic resonance (NMR) and electronic spin resonance (ESR), and magnetic resonance angioplasty (MRA). The preferred imaging modality is NMR. Of course, in addition to MRI, magnetic imaging may also be used to detect vesicles within the scope of the present invention. Magnetic imaging uses a magnetic field yet need not use pulsed gradients or radiofrequency energy. Magnetic imaging can be used to detect magnetic vesicles, such as and not limited to ferromagnetic vesicles. Magnetic imaging can be performed by a magnetometer superconducting quantum inferometry device (SQUID). SQUID permits rapid screening of all of the body tissues for the magnetic particles; the ultrasound may then be localized to those regions. In this application, magnetic resonance imaging includes magnetic imaging, while it is understood that magnetic imaging is the imaging of magnetic vesicles and does not include resonance of the nuclei thereof.

While not intending to be bound by any particular theory of operation, the present invention is believed to rely, at least in part, on the fact that gas, liquid, and solid phases have different magnetic susceptibilities. At the interface of gas and water, for example, the magnetic domains are altered and this results in dephasing of the spins of, e.g., the hydrogen nuclei. In imaging, this is seen as a decrease in signal intensity adjacent to the gas/water interface. This effect is more marked on T2 weighted images and most prominent on gradient echo pulse sequences. Using narrow bandwidth extended read-out pulse sequences increases the effect. The longer the echo time on a gradient echo pulse sequence, the greater the effect (i.e., the greater the degree and size of signal loss).

The stabilized gas-filled vesicles useful in the invention methods are believed to rely on this phase magnetic susceptibility difference, as well as on the other characteristics described in more detail herein, to act as a high performance level magnetic resonance imaging contrast medium as well as being effective in disruption of blood clots. The vesicles are formed from, i.e., created out of, a matrix of stabilizing compounds that permit the gas-filled vesicles to be established and thereafter retain their size and shape for the period of time required to be useful in magnetic resonance imaging. The compounds also permit rupture of the vesicles at a certain ultrasound energy level. These stabilizing lipid compounds are most typically those which have a hydrophobic/hydrophilic character which allows them to form monolayers or bilayers, etc., and vesicles, in the presence of water. Thus, water, saline or some other water-based medium, often referred to hereafter as a carrier, is generally an aspect of the stabilized gas-filled vesicle composition used in the invention methods.

The biocompatible stabilizing lipid may, in fact, be a mixture of compounds (e.g., lipids) that contribute various desirable attributes to the stabilized vesicles. For example, compounds that assist in the dissolution or dispersion of the fundamental stabilizing compound have been found advantageous.

A further element of the stabilized vesicles is a gas, which can be a gas at the time the vesicles are made, or can be a gaseous precursor that, responsive to an activating factor, such as temperature, is transformed from the liquid or solid phase to the gas phase.

The various aspects of the stabilized gas-filled contrast medium useful in the present invention will now be described.

Methods of Use

In another embodiment, the invention provides methods of simultaneous magnetic resonance directed noninvasive ultrasound by administering gas-filled vesicles to a patient requiring disruption of a blood clot, scanning the patient with magnetic resonance imaging to identify the region of the patient requiring lysis of a blood clot, and simultaneously applying ultrasound and magnetic resonance to the region. “Region” of a patient, means the whole vasculature or a particular area or portion of the vasculature of the patient.

After administration to a patient, the vesicles can be visualized by MRI. For example, when the location of the vesicles is determined to be in the desired region of the patient, as ascertained by MRI, then ultrasound energy using the parameters described herein, is applied to the region. The vesicles are activated by the energy, can burst (i.e., cavitate) and disrupt blood clots into micron-sized and smaller particles, thus physically lysing the blood clot to improve blood flow in the region treated. Simultaneously, the region can also be visualized by magnetic resonance imaging, if desired, to monitor the progress of thrombolysis.

The energy level that can safely be administered, using vesicles as nuclei for thrombolysis without excess heating of the vascular tissue or discomfort to the patient, is in the range from 0.1 Watts/cm² to about 30 Watts/cm², more preferably about 2 Watts/cm² to about 10 Watts/cm², depending on the region of the patient's vasculature to be treated. The duty cycle can be between 0-100%, more preferably from about 10% to about 90% or about 20% to about 80%. For example, if the blood clot is in the brain, as in the case of ischemic or hemorrhagic stroke, the ultrasound can be administered through the skull, preferably utilizing the temporal window to apply ultrasound to an effected cerebral artery while minimizing bone obstruction.

In addition to the amount of energy, an effective duty cycle of the ultrasound used for thrombolysis in the invention methods will vary depending upon the location in the body of the blood clot. Rather than a continuous wave, the ultrasound is administered as one or more pulses of energy. In general, if the energy is 10 Watts/cm², the pulse duration can be about 0.1% to about 100% of the duty cycle without overheating the vasculature or causing substantial discomfort to the patient. Alternatively, in certain regions within the body, an energy setting greater than 2 Watts/cm² to about 10 Watts/cm² can be used for about 10% to about 80% of the duty cycle.

One of skill in the art will know how to select an effective duty cycle within this range according to the particular region of the patient to which ultrasound is to be administered taking into consideration such factors as the size of the clot, the type of tissue involved (i.e., whether bone or soft tissue), and the like. In general, however, a heavily muscled or bony area will require a higher duty cycle than when the region to be treated lies under skin and the like.

Similarly, the period of time during which the ultrasound treatment is continued at the selected duty cycle to successfully accomplish thrombolysis can vary. Generally, effective thrombolysis can be accomplished within a 1 hour treatment. However, the period of treatment time can be as short as one minute or up to about 8 hours, for example about 30 minutes to about 2 hours.

In certain embodiments of the invention methods, the ultrasonic energy can be focused and the focal zone can be chosen to target the region of vesicles adjacent to the blood clot to be lysed. In other embodiments, non-focused ultrasound is employed.

In using the vesicles in the invention methods, the sound energy may be pulsed, for example in echo train lengths of at least about 8 and preferably at least about 20 pulses at a time.

Either fixed frequency or modulated frequency ultrasound may be used. Fixed frequency is defined wherein the frequency of the sound wave is constant over time. A modulated frequency is one in which the wave frequency changes over time, for example, from high to low (PRICH) or from low to high (CHIRP). For example, a PRICH pulse with an initial frequency of 10 MHz of sonic energy can sweep to 1 MHz with increasing power from 1 to 3 watts. Focused, frequency modulated, high-energy ultrasound may increase the rate of local gaseous expansion within the vesicles and rupturing to provide local lysis of a blood clot.

The frequency of the sound used may vary from about 0.025 to about 100 megahertz. Frequency ranges between about 0.75 and about 3 megahertz, for example, frequencies between about 1 and about 2 megahertz are suitable. For very small vesicles, e.g., below 0.5 micron diameter, higher frequencies of sound may be more effective as these smaller vesicles will absorb sonic energy more effectively at higher frequencies of sound. When very high frequencies are used, e.g., over 10 megahertz, the sonic energy will generally have limited depth penetration into fluids and tissues. External application may be preferred for clots near the skin and other superficial tissues, but for deep structures, the application of sonic energy via interstitial probes or intravascular ultrasound catheters may be more useful.

The energy is deposited into the tissues using a hand held ultrasound transducer, for example a magnetic resonance compatible transducer if MRI is to be used to monitor the ultrasound procedure. The ultrasound transducer is made out of non-ferrous and non-ferromagnetic material. The cables supplying energy to the ultrasound transducers may have Faraday shields to decrease the potential for artifacts, which can be caused by the electrical energy passing through the cables to supply the transducers.

Within these parameters, direct and rapid disruption of the blood clot results. Simultaneous MRI can be performed with the vesicles used to visualize the target zone or region. Then together with ultrasound, the vesicles potentiate the lysis of a blood clot in the target zone.

Rupture or activation of vesicles used in the invention methods can take place at the indicated energy range. As the vesicle is pulsed by ultrasound energy, the vesicle membrane degenerates. While there is likely a transient microdomain of increased temperature associated with the vesicle rupture, this process does not damage the surrounding tissues when energy and pulsing is applied at the indicated energy range. This effect of vesicle rupture can optionally also be advantageously used for localized delivery of a therapeutic. Thus, a therapeutic agent, such as tPA, either natural or recombinant, urokinase, pro-urokinase, reteplase, wafarins, tenecteplase, streptokinase, hirudin, or an anticoagulant such as heparin, e.g. herapin sulfate and low molecular weight heparin or nitrous oxide optionally can be released to a region of the vasculature using the invention methods.

In the case of a gaseous precursor, as ultrasound energy is focused on the precursor, the precursor will convert to the gaseous state. The enlarging gaseous void creates a domain of increasing magnetic susceptibility and is readily monitored on the magnetic resonance images. Monitoring is particularly enhanced by selecting precursors with well-defined liquid to gas conversion temperatures, such as perfluorohexane at 56° C. As the vesicles form from gaseous precursors, the materials (i.e., the vesicle) surrounding the gaseous precursor will rupture. In addition, a therapeutic agent sequestered within the vesicles can be released locally into the adjacent tissue. As the gaseous precursor converts to the gaseous state, the absorption of energy by the vesicle interface increases.

When used as a contrast medium for monitoring the progress of a treatment as described herein, the vesicles can be particularly useful in providing images of and permitting ultrasound mediated lysis of a blood clot and optional drug delivery in the cardiovascular region, but can also be employed more broadly for monitoring such aspects of the invention as drug delivery, the location of the blood clot, the infusion of vesicles, blood clot destruction, the presence and destruction of the vesicles at the region of interest in the subject, and the condition of the vessel lining.

“Cardiovascular region,” as that phrase is used herein, means the region of the patient defined by the heart and the vasculature leading directly to and from the heart. The phrase “vasculature,” as used herein, means the blood vessels (arteries, veins, etc.) in the body or in an organ or part of the body. The “patient” can be any type of mammal, but most preferably is a human.

As one skilled in the art would recognize, administration of the stabilized gas-filled vesicles used in the present invention can be carried out in various fashions, such as intravascularly, intravenously, intraarterially, and the like, using a variety of dosage forms. Additionally, the vesicles can be administered locally by injection when the region to be treated is known. When the region to be treated is the cardiovascular region, administration of the contrast medium of the invention is preferably carried out intravascularly. The useful and “effective amount” of the vesicles administered or the various drugs contemplated for use in the invention methods and the particular mode of administration will vary depending upon the size of the blood clot, the age, weight and the particular mammal to be treated, and the vascular region thereof to be treated as well as the particular vesicles of the invention to be employed. Typically, dosage is initiated at lower levels and increased until the desired effect is achieved, e.g. blood clot lysis or contrast enhancement. Various combinations of the stabilized gas-filled vesicles can be used to modify the relaxation behavior of the medium or to alter properties, such as the viscosity, osmolarity, and the like.

In carrying out noninvasive ultrasound methods of the present invention, the gas or gaseous precursor-filled vesicles can be used alone, or in combination with other diagnostic, therapeutic or other agents. Such other agents include excipients, such as flavoring or coloring materials. When magnetic resonance imaging is employed as described herein, the techniques used are conventional and are well described, for example, in D. M. Kean and M. A. Smith, Magnetic Resonance Imaging: Principles and Applications, (William and Wilkins: Baltimore 1986). Contemplated MRI techniques include, but are not limited to, nuclear magnetic resonance (NMR) and electronic spin resonance (ESR). The preferred imaging modality is NMR.

By “ultrasound mediated lysis of a blood clot” or “thrombolysis,” as the terms are used herein, is meant lysis or disruption of a thrombus or blood clot within the vasculature and the activation or rupture of vesicles adjacent to the blood clot by ultrasonic energy.

Gases and Gaseous Precursors

The vesicles of the invention encapsulate a gas or gaseous precursor. The term “gas-or gaseous precursor-filled”, as used to describe the vesicles used in the invention methods, means that the vesicles have an interior volume that is comprised of at least about 10% gas or gaseous precursor, preferably at least about 25% gas or gaseous precursor, more preferably at least about 50% gas or gaseous precursor, even more preferably at least about 75% gas or gaseous precursor, and most preferably at least about 90% gas or gaseous precursor. In use, where the presence of gas is important, it is preferred that the interior vesicle volume comprise at least 10% gas, preferably at least about 25%, 50%, 75%, and most preferably at least 90% gas.

Select biocompatible gas or gaseous precursors can be used to form the stabilized gas- or gaseous precursor-filled vesicles used in the invention methods. By “biocompatible” is meant a gas or gaseous precursor that, when introduced into the blood of a human patient, will not result in any degree of unacceptable toxicity, including allergenic responses and disease states, and preferably is inert. Such gases include, for example, various fluorinated gaseous compounds, such as various perfluorocarbon, hydrofluorocarbon, and sulfur hexafluoride gases can be utilized in the preparation of the gas-filled vesicles. Further, paramagnetic gases or gases such as ¹⁷O can be used; however, the oxygen should be stabilized, since oxygen gas is soluble in blood.

Of all of the gases, perfluorocarbons containing less than 10 carbons are preferred due to their low (limited) solubility and diffusability in aqueous media. Such gases are also easier to stabilize into the form of bubbles in aqueous media due to these properties. Suitable perfluorocarbon gases include, for example, perfluorobutane, perfluorocyclobutane, perfluoromethane, perfluoroethane, perfluoropropane, and perfluoropentane, perfluorohexane, most preferably perfluoropropane. A mixture of different types of gases, such as a perfluorocarbon gas and another type of gas such as oxygen, can also be used. Indeed, it is believed that a combination of gases can be particularly useful in simultaneous magnetic resonance directed noninvasive ultrasound applications.

The gaseous precursors can be in the form of a liquid or solid. Solid and liquid gaseous precursors are activated to the gaseous state by the ultrasonic energy administered. The use of gaseous precursors is an optional embodiment of the present invention. In particular, perfluorocarbons containing less than 10 carbons have been found to be suitable for use as gaseous precursors, i.e., in the liquid or solid state. Whether such a perfluorocarbon is a gas, liquid, or solid depends, of course, on its liquid/gas or solid/gas phase transition temperature, or boiling point. For example, one of the more preferred perfluorocarbons is perfluoropentane, which has a liquid/gas phase transition temperature or boiling point of 27° C., which means that it will be a liquid at ordinary room temperature, but will become a gas in the environment of the human body, where the temperature will be above its liquid/gas phase transition temperature or boiling point. Thus, under normal circumstance, perfluoropentane is a gaseous precursor and during transition is a mixture of gas or gaseous precursor. All of these conditions are meant to be included by the phrase “gas or gaseous precursor”. As further examples, there are perfluorobutane and perfluorohexane, the next closest homologs of perfluoropentane. The liquid/gas phase transition temperature of perfluorobutane is 4° C. and that of perfluorohexane is 57° C., making the former potentially a gaseous precursor, but generally more useful as a gas, while the latter would generally be a gaseous precursor, except under unusual circumstances, because of its high boiling point. Solid and liquid gaseous precursors can, in many instances, be activated to the gaseous state by the ultrasonic energy administered.

For example, perflutren (octafluoropropane) lipid microspheres (Bristol-Myers Squibb; Definity™) is an ultrasound contrast agent approved for use in certain related diagnostic purposes. Perflutren lipid emulsion may be administered by either an intravenous bolus or infusion. The recommended bolus dose is 10 microliters/kilogram (kg) of the activated product within 30 to 60 seconds, followed by a 10 milliliter (mL) saline flush. If necessary, a second 10 microliter/kg dose followed by a second 10 mL saline flush may be administered 30 minutes after the first injection to prolong contrast enhancement. Alternatively, the recommended dose via intravenous infusion is 1.4 milliliters (mL)(or 10 mL/kg in divided doses) added to 50 mL of preservative-free saline. The rate of infusion can be initiated at 4 mL/minute and titrated as needed to achieve optimal image enhancement, not to exceed 10 mL/minute.

Another aspect of the present invention is the optional inclusion in the vesicles of an additional fluorinated compound as a stabilizing agent, especially a perfluorocarbon compound, which will be in the liquid state at the temperature of use of the vesicles, to assist or enhance the stability of the gas or gaseous precursor filled vesicles. Such additional fluorinated compounds include various liquid fluorinated compounds, such as fluorinated surfactants manufactured by the DuPont Company (Wilmington, Del.), e.g., ZONYL®., as well as liquid perfluorocarbons. The fluorinated compounds can be perfluorocarbons. Suitable perfluorocarbons useful as additional stabilizing agents include perfluorooctylbromide (PFOB), per-fluorodecalin, perfluorododecalin, perfluorooctyliodide, perfluorotripropylamine, and perfluorotributylamine. In general, perfluorocarbons over six carbon atoms in length will not be gaseous, i.e., in the gas state, but rather will be liquids, i.e., in the liquid state, at normal human body temperature. These compounds may, however, additionally be utilized in preparing the stabilized gas or gaseous precursor filled vesicles used in the present invention. For example, the additional stabilizing agent can be perfluorooctylbromide or perfluorohexane, which is in the liquid state at room temperature. The gas that is present can be, e.g., nitrogen or perfluoropropane, or can be derived from a gaseous precursor, which may also be a perfluorocarbon, e.g., perfluoropentane. In that case, the vesicles of the present invention would be prepared from a mixture of perfluorocarbons, which for the examples given would be perfluoropropane (gas) or perfluoropentane (gaseous precursor) and perfluorooctylbromide (liquid). Although not intending to be bound by any theory, it is believed that the liquid fluorinated compound partitions to the interface between the gas and the membrane surface of the vesicle. There is thus formed a further stabilizing layer of liquid fluorinated compound on the internal surface of the stabilizing compound, e.g., a biocompatible lipid used to form the vesicle, and this perfluorocarbon layer also serves the purpose of preventing the gas from diffusing through the vesicle membrane. Thus, it is within the scope of the present invention to utilize a gas or gaseous precursor, such as a perfluorocarbon gaseous precursor, e.g., perfluoropentane, together with a perfluorocarbon that remains liquid after administration to a patient, i.e., whose liquid to gas phase transition temperature is above the body temperature of the patient, e.g., perfluorooctylbromide or perfluorohexane.

The size of the gas or gaseous precursor filled vesicles becomes stabilized when the stabilizing compounds described herein are employed; and the size of the vesicles can then be adjusted for the particular intended end use. For example, thrombolysis may require vesicles that are no larger than about 1 micron to no larger than about 12 microns in average diameter, for example, from about 1 to about 4 microns or about 1.1 to about 3.3 microns (in vitro average diameter measurements)—smaller than a red blood cell (6-8 microns). The size of the gas-filled vesicles can be adjusted, if desired, by a variety of procedures including microemulsification, vortexing, extrusion, filtration, sonication, homogenization, repeated freezing and thawing cycles, extrusion under pressure through pores of defined size, and similar methods.

As noted above, the embodiments of the present invention may also include, with respect to their preparation, formation and use, gaseous precursors that can be activated by temperature. Further below is set out Table I listing a series of gaseous precursors that undergo phase transitions from liquid to gaseous states at relatively close to normal body temperature (37° C.) or below, and the size of the emulsified droplets that would be required to form a micro bubble of a maximum size of 10 microns.

TABLE 1
Physical Characteristics of Gaseous Precursors and
Diameter of Emulsified Droplet to Form a 10μ Vesicle*

| | | | | Diameter (μ) of |
|---|---|---|---|---|
| | | | | Emulsified |
| | Molecular | Boiling Point | | Droplets to Make |
| Perfluoro Compound | Weight | °(C.) | Density | 10 Micron Vesicle |
| pentane 1-(isopentane) | 288.04 | 28.5 | 1.7326 | 2.9 |
| pentane 1-fluorobutane | 76.11 | 32.5 | 6.7789 | 1.2 |
| 2-methyl butan (isopentane) | 72.15 | 27.8 | 0.6201 | 2.6 |
| 2-methyl 1-butane | 70.13 | 31.2 | 0.6504 | 2.5 |
| 2-methyl-2-butane | 70.13 | 38.6 | 0.6623 | 2.5 |
| 1-butene-3-yne-2-methyl | 66.10 | 34.0 | 0.6801 | 2.4 |
| 3-methyl-1-butyne | 68.12 | 29.5 | 0.6660 | 2.5 |
| octafluoro cyclobutane | 200.04 | −5.8 | 1.48 | 2.8 |
| decafluoro butane | 238.04 | −2 | 1.517 | 3.0 |
| hexafluoro ethane | 138.01 | −78.1 | 1.607 | 2.7 |

*Source: Chemical Rubber Company Handbook of Chemistry and Physics, Robert C. Weast and David R. Lide, eds., CRC Press, Inc. Boca Raton, Florida (1989-1990).

There is also set out below a list composed of suitable potential gaseous precursors that can be used to form vesicles of defined size. However, the list is not intended to be limiting, since it is possible to use other gaseous precursors for that purpose. In fact, for a variety of different applications, virtually any liquid can be used to make gaseous precursors so long as it is biocompatible and capable of undergoing a phase transition to the gas phase upon passing through the appropriate temperature, so that at least at some point in use it provides a gas. Suitable gaseous precursors for use in the present invention are the following: hexafluoro acetone, isopropyl acetylene, allene, tetrafluoro-allene, boron trifluoride, isobutane, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluoro butane, 2-methyl-butane, decafluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluoro butyne, 3-methyl-1-butyne, perfluoro-2-butyne, 2-bromo-butyraldehyde, carbonyl sulfide, crotononitrile, cyclobutane, methyl-cyclobutane, octafluoro-cyclobutane, perfluoro cyclobutene, 3-chlorocyclopentene, octafluorocyclopentene, cyclopropane, 1,2-dimethyl cyclopropane, 1,1-dimethylcyclopropane, 1,2-dimethyl-cyclopropane, ethylcyclopropane, methylcyclopropane, diacetylene, 3-ethyl-3-methyl diaziridine, 1,1,1-trifluorodiazoethane, dimethyl amine, hexafluorodimethylamine, dimethylethylamine, bis (dimethylphosphine)amine, perfluorohexane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropentafluoroethane, dichlorotrifluoroethane, fluoroethane, hexafluoroethane, nitropentafluoroethane, nitrosopentafluoroethane, perfluoroethylamine, ethyl vinyl ether, 1,1-dichloroethane, 1,1-dichloro-1,2-difluoroethane, 1,2-difluoroethane, methane, trifluoromethanesulfonylchloride, trifluoromethanesulfonylfluoride, bromodifluoronitrosomethane, bromofluoromethane, bromochlorofluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodomethane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsulfide, methyl vinyl ether, neon, neopentane, nitrogen (N.sub.2), nitrous oxide, 1,2,3-nonadecane-tricarboxylic acid-2-hydroxytrimethylester, 1-nonene-3-yne, oxygen (O₂), 1,4-pentadiene, n-pentane, perfluoropentane, 4-amino-4-methylpentan-2-one, 1-pentene, 2-pentene (cis), 2-pentene (trans), 3-bromopent-1-ene, perfluoropent-1-ene, tetrachlorophthalic acid, 2,3,6-trimethylpiperidine, propane, 1,1,1,2,2,3-hexafluoropropane, 1,2-epoxypropane, 2,2-difluoropropane, 2-aminopropane, 2-chloropropane, heptafluoro-1-nitropropane, heptafluoro-1-nitrosopropane, perfluoropropane, propene, hexafluoropropane, 1,1,1,2,3,3-hexafluoro-2,3 dichloropropane, 1-chloropropane, chloropropane-(trans), 2-chloropropane, 3-fluoropropane, propyne, 3,3,3-trifluoropropyne, 3-fluorostyrene, sulfur hexafluoride, sulfur (di)-decafluoride (S₂ F₁₀), 2,4-diaminotoluene, trifluoroacetonitrile, trifluoromethyl peroxide, trifluoromethyl sulfide, tungsten hexafluoride, vinyl acetylene, vinyl ether, and xenon.

The perfluorocarbons containing less than 10 carbon atoms, as already indicated, are preferred for use as the gas or gaseous precursors, as well as additional stabilizing components. Included in such perfluorocarbon compositions are saturated perfluorocarbons, unsaturated perfluorocarbons, and cyclic perfluorocarbons. Examples of suitable saturated perfluorocarbons are the following: tetrafluoromethane, hexafluoroethane, octafluoropropane, decafluorobutane, dodecafluoropentane, perfluorohexane, and perfluoroheptane. Cyclic perfluorocarbons, which have the formula CₙF₂ₙ, where n is from 3 to 8, preferably 3 to 6, may also be preferred, and include, e.g., hexafluorocyclopropane, octafluorocyclobutane, and decafluorocyclopentane. Mono-hydrogenated versions of these compounds and 2-hydroheptafluoropropane are also useful.

It is part of the present invention to optimize the utility of the vesicles by using gases of limited solubility. By limited solubility, is meant limited ability of the gas to diffuse out of the vesicles by virtue of its solubility in the surrounding aqueous medium (e.g., blood). A greater solubility in the aqueous medium imposes a gradient with the gas in the vesicle such that the gas will have a tendency to diffuse out of the vesicle. Therefore, in one aspect, the gas entrapped in the vesicle has solubility less than that of oxygen, i.e., 1 part gas in 32 parts water (See Matheson Gas Data Book, 1966, Matheson Company Inc.), less than that of air, or less than that of nitrogen.

Stabilizing Compounds

One or more biocompatible lipid stabilizing compounds are employed to form the vesicles, and to assure continued encapsulation of the gases or gaseous precursors until the vesicles have reached the region of the vasculature where the blood clot is located. Even for relatively insoluble, non-diffusible gases such as perfluoropropane or sulfur hexafluoride, improved vesicle preparations are obtained when one or more stabilizing compounds are utilized in the formation of the gas or gaseous precursor filled vesicles. These compounds maintain the stability and the integrity of the vesicles with regard to their size, shape and other attributes.

The terms “stable” or “stabilized”, as used herein, means that the vesicles are substantially resistant to degradation, i.e., are resistant to the loss of vesicle structure or encapsulated gas or gaseous precursor for a useful period of time. Typically, the vesicles of the invention have a good shelf life, often retaining at least about 90 percent by volume of its original volume for a period of at least about two or three weeks under normal ambient conditions, although the shelf life can be at least a month up to about three years, for example two, or six or eighteen months. Thus, the gas- or gaseous precursor-filled vesicles typically have a good shelf life, sometimes even under adverse conditions, such as temperatures and pressures above or below those experienced under normal ambient conditions. However, because of the ease of formulation, i.e., the ability to produce the vesicles just prior to administration, these vesicles can be conveniently made on site.

Biocompatible Lipids and Polymers

The lipids and polymers employed in preparing the vesicles of the invention are biocompatible. By “biocompatible” is meant a lipid or polymer which, when introduced into the blood of a human patient, will not result in any degree of unacceptable toxicity, including allergenic responses and disease states. Preferably the lipids are inert.

Such lipid materials can be what is often referred to as “amphiphilic” in nature, by which is meant any composition of matter which has, on the one hand, lipophilic, i.e., hydrophobic properties, while on the other hand, and at the same time, having lipophobic, i.e., hydrophilic properties. Hydrophilic groups can be charged moieties or other groups having an affinity for water. Natural and synthetic phospholipids are examples of amphiphilic lipids useful in preparing the stabilized vesicles used in the invention methods. Phospholipids, which contain charged phosphate “head” groups attached to long hydrocarbon tails, can form a single bilayer (unilamellar) arrangement in which all of the water-insoluble hydrocarbon tails are in contact with one another, leaving the highly charged phosphate head regions free to interact with a polar aqueous environment. A series of concentric bilayers are possible, i.e., oligolamellar and multilamellar vesicles, and such arrangements are also contemplated to be an aspect of the stabilizing agents used in preparation of the vesicles. The ability to form such bilayer arrangements is one feature of the lipid materials useful in the present invention.

The lipid may alternatively be in the form of a monolayer, and the monolayer lipids can be used to form a single monolayer (unilamellar) arrangement or a series of concentric monolayers, i.e., oligolamellar or multilamellar vesicles. Such lipid arrangements are also considered to be within the scope of the invention.

It has also been found advantageous to prepare the vesicles at a temperature below the gel to liquid crystalline phase transition temperature of a lipid(s) used as the stabilizing compound. This phase transition temperature is the temperature at which a lipid bilayer will convert from a gel state to a liquid crystalline state. See, for example, Chapman et al, J. Biol. Chem. (1974) 249:2512-2521. Generally, the higher the gel/liquid phase transition temperature, the more impermeable the gas or gaseous precursor filled vesicles are at any given temperature. (See Derek Marsh, CRC Handbook of Lipid Bilayers (CRC Press, Boca Raton, Fla. 1990), at p. 139 for main chain melting transitions of saturated diacyl-sn-glycero-3-phosphocholines). The gel/liquid crystalline state phase transition temperatures of various lipids will be readily apparent to those skilled in the art and are described, for example, in Gregoriadis, ed., Liposome Technology, Vol. I, 1-18 (CRC Press, 1984). Table 2, below, lists some of the representative lipids and their phase transition temperatures:

TABLE 2
Saturated Diacyl sn-Glycero(3)Phosphocholines:
Main Chain Phase Transition Temperatures*

| | Main Phase Transition |
|---|---|
| Carbons in Acyl Chains | Temperature ° C. |
| 1,2-(12:0) | −1.0 |
| 1,2-(13.0) | 13.7 |
| 1,2-(14:0) | 23.5 |
| 1,2-(15:0) | 34.5 |
| 1,2-(16:0) | 41.4 |
| 1,2-(17:0) | 48.2 |
| 1,2-(18:0) | 55.1 |
| 1,2-(19:0) | 61.3 |
| 1,2-(20:0) | 64.5 |
| 1,2-(21:0) | 71.1 |
| 1,2-(22:0) | 74.0 |
| 1,2-(23:0) | 79.5 |
| 1,2-(24:0) | 80.1 |

*Derek Marsh, “CRC Handbook of Lipid Bilayers”, CRC Press, Boca Raton, Florida (1990), page 139.

In particular, it has been found possible to enhance the stability of the vesicles used in the present invention by incorporating at least a small amount, i.e., about 1 to about 10 mole percent of the total lipid, of a negatively charged lipid into the lipid from which the gas or gaseous precursor filled vesicles are to be formed. Suitable negatively charged lipids include, e.g., phosphatidylserine, phosphatidic acid, and fatty acids. Such negatively charged lipids provide added stability by counteracting the tendency of the vesicles to rupture by fusing together, i.e., by establishing a uniform negatively charged layer on the outer surface of the vesicle that is repulsed by a similarly charged outer layer on the other vesicles. In this way, the vesicles will tend to be prevented from touching, which would often lead to membrane rupture and consolidation of the contacting vesicles into a single, larger vesicle. A continuation of this process of consolidation would lead to significant degradation of the vesicles.

The lipid material or other stabilizing compound used to form the vesicles is also preferably flexible, by which is meant, in the context of gas or gaseous precursor filled vesicles, the ability of a structure to alter its shape, for example, in order to pass through an opening having a size smaller than the vesicle.

In selecting a lipid for preparing the stabilized vesicles used in the present invention, a wide variety of lipids will be found to be suitable for their construction. Particularly useful are any of the materials or combinations thereof known to those skilled in the art as suitable for liposome preparation. The lipids used can be of natural, synthetic, or semi-synthetic origin.

Lipids useful in preparing the gas or gaseous precursor filled vesicles used in the invention include methods, include, but are not limited to: lipids such as fatty acids, lysolipids, phosphatidylcholine with both saturated and unsaturated lipids including dioleoylphosphatidylcholine; dimyristoylphosphatidylcholine; dipentadecanoylphosphatidylcholine; dilauroylphosphatidylcholine; dipalmitoylphosphatidylcholine (DPPC); distearoyl-phosphatidylcholine (DSPC); phosphatidylethanolamines such as dioleoylphosphatidylethanolamine and dipalmitoyl-phosphatidylethanolamine (DPPE); phosphatidylserine; phosphatidylglycerol; phosphatidylinositol; sphingolipids such as sphingomyelin; glycolipids such as ganglioside GM1 and GM2; glucolipids; sulfatides; glycosphingolipids; phosphatidic acids such as dipalymitoylphosphatidic acid (DPPA); palmitic acid; stearic acid; arachidonic acid; oleic acid; lipids bearing polymers such as polyethylene glycol, i.e., PEGylated lipids, chitin, hyaluronic acid or polyvinylpyrrolidone; lipids bearing sulfonated mono-, di-, oligo- or polysaccharides; cholesterol, cholesterol sulfate and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of 6-8 carbons in length; synthetic phospholipids with asymmetric acyl chains (e.g., with one acyl chain of 6 carbons and another acyl chain of 12 carbons); ceramides; non-ionic liposomes including niosomes such as polyoxyethylene fatty acid esters, polyoxyethylene fatty alcohols, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene polymers, and polyoxyethylene fatty acid stearates; sterol aliphatic acid esters including cholesterol sulfate, cholesterol butyrate, cholesterol iso-butyrate, cholesterol palmitate, cholesterol stearate, lanosterol acetate, ergosterol palmitate, and phytosterol n-butyrate; sterol esters of sugar acids including cholesterol glucuroneide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid, accharic acid, and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; longchain alcohols including n-decyl alcohol, lauryl alcohol, myristyl alcohol, cetyl alcohol, and n-octadecyl alcohol; 6-(5-cholesten-3.beta.-yloxy)-1-thio-.beta.-D-galactopyranoside; digalactosyldiglyceride; 6-(5-cholesten-3.beta.-yloxy)hexyl-6-amino-6-deoxy-1-thio-.beta.-D-galacto pyranoside; 6-(5-cholesten-3.beta.-yloxy)hexyl-6-amino-6-deoxyl-1-thio-.alpha.-D-manno pyranoside; 12-(((7′-diethylaminocoumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoylphosphatidylethanol-amine; 1,2-dioleoyl-sn-glycerol;1,2-dipalmitoyl-sn-3-succinylglycerol; 1,3-dipalmitoyl-2-succinylglycerol; 1-hexadecyl-2-palmitoyl-glycerophosphoethanolamine and palmitoylhomocysteine, and combinations thereof.

If desired, a variety of cationic lipids such as DOTMA, N-[1-(2,3-dioleoyloxy)propyl]-N,N,N-trimethylammoium chloride; DOTAP, 1,2-dioleoyloxy-3-(trimethylammonio)propane; and DOTB, 1,2-dioleoyl-3-(4′-trimethyl-ammonio)butanoyl-sn-glycerol can be used. In general the molar ratio of cationic lipid to non-cationic lipid in the liposome can be, for example, 1:1000, 1:100, or between 2:1 to 1:10, for example, in the range from about 1:1 to about 1:2. A wide variety of lipids may comprise the non-cationic lipid when cationic lipid is used to construct the vesicle. Examples of a non-cationic lipid include, for example dipalmitoylphosphatidylcholine, dipalmitoylphosphatidylethanolamine or dioleoylphosphatidyl-ethanolamine. In lieu of cationic lipids as described above, lipids bearing cationic polymers such as polylysine or polyarginine, as well as alkyl phosphonates, alkyl phosphinates, and alkyl phosphites, may also be used to construct the vesicles.

The most preferred lipids are phospholipids, such as di-palmitoylphosphatidyl choline (DPPC); 1,2-dipalmitoyl-sn-glycero-3-phosphoethanolamine (DPPE); Diphenylphosphoryl azide (DPPA); and distearoylphospatidylcholin (DSPC).

In addition, examples of saturated and unsaturated fatty acids that can be used to prepare the stabilized vesicles used in the present invention, in the form of gas or gaseous precursor filled mixed micelles, can include molecules containing from 12 carbon atoms and to 22 carbon atoms in either linear or branched form. Hydrocarbon groups consisting of isoprenoid units and prenyl groups can be used as well. Examples of saturated fatty acids that are suitable include, but are not limited to, lauric, myristic, palmitic, and stearic acids; examples of unsaturated fatty acids that can be used include, but are not limited to, lauroleic, physeteric, myristoleic, palmitoleic, petroselinic, and oleic acids; examples of branched fatty acids that can be used include, but are not limited to, isolauric, isomyristic, isopalmitic, and isostearic acids. In addition to the saturated and unsaturated groups, gas or gaseous precursor filled mixed micelles can also be composed of 5 carbon isoprenoid and prenyl groups. In addition, partially fluorinated phospholipids can be used as stabilizing compounds for coating the vesicles.

In one embodiment of the invention methods, the stabilizing compound from which the stabilized gas or gaseous precursor filled vesicles are formed comprises three biocompatible lipids: (1) a neutral (e.g., nonionic or zwitterionic) lipid, (2) a negatively charged lipid, and (3) a lipid bearing a hydrophilic polymer. Usually, the amount of the negatively charged lipid will be greater than 1 mole percent of total lipid present, and the amount of lipid bearing a hydrophilic polymer can be greater than 1 mole percent of total lipid present. For example, the negatively charged lipid can be a phosphatidic acid. In another example, the lipid bearing a hydrophilic polymer can be a lipid covalently bound to the polymer, and the polymer will have a weight average molecular weight of from about 400 to about 100,000. Hydrophilic polymers particularly suitable for use in this case, include polyethyleneglycol (PEG), polypropyleneglycol, polyvinylalcohol, and polyvinylpyrrolidone and copolymers thereof. The PEG or other polymer can be bound to the DPPE or other lipid through a covalent linkage, such as through an amide, carbamate or amine linkage. Alternatively, ester, ether, thioester, thioamide or disulfide (thioester) linkages can be used with the PEG or other polymer to bind the polymer to, for example, cholesterol or other phospholipids. Where the hydrophilic polymer is polyethyleneglycol, a lipid bearing such a polymer will be said to be “PEGylated.” An example of a lipid bearing a hydrophilic polymer is dipalmitoylphosphatidylethanolamine-polyethyleneglycol 5000, i.e., a dipalmitoylphosphatidylethanolamine lipid having a polyethyleneglycol polymer of a mean weight average molecular weight of about 5000 attached thereto (DPPE-PEG5000); or distearoyl-phosphatidylethanolamine-polyethyleneglycol 5000.

In various embodiments, the vesicles contemplated by the present invention would include, e.g, 77.5 mole percent dipalmifoylphophatidylcholine (DPPC), with 12.5 mole percent of dipalmitoylphosphatidic acid (DPPA), and with 10 mole percent of dipalmitoylphosphatidylethanolamine-polyethyleneglycol-5000 (DPPE/PEG5000). These compositions can have an 82/10/8 ratio of mole percentages, respectively. The DPPC component is effectively neutral, since the phosphtidyl portion is negatively charged and the choline portion is positively charged. Consequently, the DPPA component, which is negatively charged, is added to enhance stabilization in accordance with the mechanism described further above regarding negatively charged lipids as an additional agent. The third component, DPPE/PEG, provides a PEGylated material bound to the lipid membrane or skin of the vesicle by the DPPE moiety, with the PEG moiety free to surround the vesicle membrane or skin, and thereby form a physical barrier to various enzymatic and other endogenous agents in the body whose function is to degrade such foreign materials. It is also theorized that the PEGylated material, because of its structural similarity to water, is able to defeat the action of the macrophages of the human immune system, which would otherwise tend to surround and remove the foreign object. The result is an increase in the time during which the stabilized vesicles can function in vivo.

It has been found that the gas or gaseous precursor filled vesicles used in the present invention can be controlled according to size, solubility and heat stability by choosing from among the various additional or auxiliary stabilizing agents described herein. These agents can affect these-parameters of the vesicles not only by their physical interaction with the lipid coatings, but also by their ability to modify the viscosity and surface tension of the surface of the gas- or gaseous precursor-filled vesicle. Accordingly, the gas or gaseous precursor filled vesicles used in the present invention can be favorably modified and further stabilized, for example, by the addition of one or more of a wide variety of (a) viscosity modifiers, including, but not limited to carbohydrates and their phosphorylated and sulfonated derivatives; and polyethers, for example, with molecular weight ranges between 400 and 1 00,000; di- and trihydroxy alkanes and their polymers, for example, with molecular weight ranges between 200 and 50,000, and propylene glycol; (b) emulsifying and solubilizing agents may also be used in conjunction with the lipids to achieve desired modifications and further stabilization; such agents include, but are not limited to, cholesterol, diethanolamine, glyceryl monostearate, lanolin alcohols, lecithin, mono- and di-glycerides, mono-ethanolamine, oleic acid, oleyl alcohol, poloxamer (e.g., poloxamer 188, poloxamer 184, and poloxamer 181), polyoxyethylene 50 stearate, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax; (c) suspending and viscosity-increasing agents that can be used with the lipids include, but are not limited to, carbomer 934P, carboxymethylcellulose, calcium and sodium and sodium 12, cellulose, dextran, gelatin, hydroxyethyl cellulose, hydroxypropyl methylcellulose, methylcellulose, propylene glycol, polyethylene oxide, povidone, alpha-d-gluconolactone, glycerol and mannitol; (d) synthetic suspending agents may also be utilized such as polyethyleneglycol (PEG), polyvinylpyrrolidone (PVP), polyvinylalcohol (PVA), polypropylene glycol, and polysorbate; and (e) tonicity raising agents can be included; such agents include but are not limited to sorbitol, propyleneglycol and glycerol.

Aqueous Diluents

As mentioned earlier, where the vesicles are lipid in nature, a particularly desired component of the stabilized vesicles is an aqueous environment of some kind, which induces the lipid, because of its hydrophobic/hydrophilic nature, to form vesicles, the most stable configuration in such an environment. The diluents which can be employed to create such an aqueous environment include, but are not limited to, water, either deionized or containing any number of non-toxic dissolved salts that do not interfere with creation and maintenance of the stabilized vesicles or their use as MRI contrast agents; and normal saline and physiological saline.

Paramagnetic and Superparamagnetic Contrast Agents

In a further embodiment of the present invention, the stabilized gas- or gaseous precursor-filled vesicles used in the invention methods may optionally further comprise additional contrast agents, such as conventional contrast agents, that serve to increase the efficacy of the vesicles for simultaneous magnetic resonance directed noninvasive ultrasound. Many such contrast agents are well known to those skilled in the art and include paramagnetic and superparamagnetic contrast agents.

Exemplary paramagnetic contrast agents suitable for encapsulation in the vesicles include stable free radicals (such as, for example, stable nitroxides), as well as compounds comprising transition, lanthanide and actinide elements, which may, if desired, be in the form of a salt or can be covalently or noncovalently bound to complexing agents (including lipophilic derivatives thereof) or to proteinaceous macromolecules.

Preferable transition, lanthanide and actinide elements include Gd(III), Mn(II), Cu(II), Cr(III), Fe(II), Fe(III), Co(II), Er(II), Ni(II), Eu(III) and Dy(III). More preferably, the elements include Gd(III), Mn(II), Cu(II), Fe(II), Fe(III), Eu(III) and Dy(III), especially Mn(II) and Gd(III).

These elements may, if desired, be in the form of a salt, such as a manganese salt, e.g., manganese chloride, manganese carbonate, manganese acetate, and organic salts of manganese such as manganese gluconate and manganese hydroxylapatite; and such as an iron salt, e.g., iron sulfides and ferric salts such as ferric chloride.

These elements may also, if desired, be bound, e.g., covalently or noncovalently, to complexing agents (including lipophilic derivatives thereof) or to proteinaceous macromolecules. Suitable complexing agents include, for example, diethylenetriamine-pentaacetic acid (DTPA), ethylene-diaminetetraacetic acid (EDTA), 1,4,7,10-tetraazacyclododecane-N,N′,N″,N′″-tetraacetic acid (DOTA), 1,4,7,10-tetraazacyclododecane-N,N′,N″-triacetic acid (DO3A), 3,6,9-triaza-12-oxa-3,6,9-tricarboxymethylene-10-carboxy-13-phenyl-trideca noic acid (B-19036), hydroxybenzylethylene-diamine diacetic acid (HBED), N,N′-bis(pyridoxyl-5-phosphate)ethylene diamine, N,N′-diacetate (DPDP), 1,4,7-triazacyclononane-N,N′N″-triacetic acid (NOTA), 1,4,8,11-tetraazacyclotetradecane-N,N′N″,N′″-tetraacetic acid (TETA), kryptands (that is, macrocyclic complexes), and desferrioxamine. Alternatively, the complexing agents can be EDTA, DTPA, DOTA, DOPA and kryptands. Lipophilic complexes thereof include alkylated derivatives of the complexing agents EDTA, DOTA, etc., for example, EDTA-DDP, that is, N,N′-bis-(carboxy-decylamidomethyl-N-2,3-dihydroxypropyl)-ethylenediamine-N,N′-diacetate; EDTA-ODP, that is N,N′-bis-(carboxy-octadecylamido-methyl-N-2,3-dihydroxypropyl)-ethylenedia mine-N,N′-diacetate; EDTA-LDP N,N′-Bis-(carboxy-laurylamidomethyl-N-2,3-dihydroxypropyl)-ethylenediamine -N,N′-diacetate; etc.; such as those described in U.S. Pat. No. 5,312,617 the disclosure of which is hereby incorporated by reference in its entirety. Suitable proteinaceous macromolecules include albumin, collagen, polyarginine, polylysine, polyhistidine, gamma-globulin and beta-globulin.

Suitable complexes thus include Mn(II)-DTPA, Mn(II)-EDTA, Mn(II)-DOTA, Mn(II)-DO3A, Mn(II)-kryptands, Gd(III)-DTPA, Gd(III)-DOTA, Gd(III)-DO3A, Gd(III)-kryptands, Cr(III)-EDTA, Cu(II)-EDTA, or iron-desferrioxamine, especially Mn(II)-DTPA or Gd(III)-DTPA.

Paramagnetic chelates, such as alkylated chelates of paramagnetic ions, as disclosed in U.S. Pat. No. 5,312,617, the disclosure of which is incorporated herein by reference in its entirety, paramagnetic copolymeric chelates as in U.S. Pat. No. 5,385,719 useful for attaching to gas-filled liposomes and to the surface of gas-filled polymeric liposomes, nitroxide stable free radicals (NSFRs) useful for attaching to lipids in gas-filled liposomes as well as to polymers for construction of gas-filled liposomes and hybrid complexes comprised of chelate moieties containing one or more paramagnetic ions in close proximity with one or more NSFRs as outlined in U.S. Pat. No. 5,407,657, can be used for constructing paramagnetic gas-filled liposomes. These hybrid complexes have greatly increased relaxivity and, therefore, increase the sensitivity to the vesicle to magnetic resonance. Nitroxides are paramagnetic contrast agents that increase both T1 and T2 relaxation rates by virtue of one unpaired electron in the nitroxide molecule. The paramagnetic effectiveness of a given compound as an MRI contrast agent is at least partly related to the number of unpaired electrons in the paramagnetic nucleus or molecule, specifically to the square of the number of unpaired electrons. For example, gadolinium has seven unpaired electrons and a nitroxide molecule has only one unpaired electron; thus gadolinium is generally a much stronger MRI contrast agent than a nitroxide. However, effective correlation time, another important parameter for assessing the effectiveness of contrast agents, confers potential increased relaxivity to the nitroxides. When the effective correlation time is very close to the proton Larmour frequency, the relaxation rate may increase dramatically. When the tumbling rate is slowed, e.g., by attaching the paramagnetic contrast agent to a large structure, it will tumble more slowly and thereby more effectively transfer energy to hasten relaxation of the water protons. In gadolinium, however, the electron spin relaxation time is rapid and will limit the extent to which slow rotational correlation times can increase relaxivity. For nitroxides, however, the electron spin correlation times are more favorable and slowing the rotational correlation time of these molecules can attain tremendous increases in relaxivity. The gas-filled vesicles used in the invention are ideal for attaining the goals of slowed rotational correlation times and resultant improvement in relaxivity. Although not intending to be bound by any particular theory of operation, it is contemplated that since the nitroxides can be designed to coat the perimeters of the gas-filled vesicles, e.g., by making alkyl derivatives thereof, the resulting correlation times can be optimized. Moreover, the resulting contrast medium of the present invention can be viewed as a magnetic sphere, a geometric configuration that maximizes relaxivity.

If desired, the nitroxides can be alkylated or otherwise derivatized, such as the nitroxides 2,2,5,5-tetramethyl-1-pyrrolidinyloxy, free radical, and 2,2,6,6-tetramethyl-1-piperidinyloxy, free radical (TMPO).

Exemplary superparamagnetic contrast agents suitable for inclusion in the gas-filled vesicles used in the invention include metal oxides and sulfides which experience a magnetic domain, ferro- or ferrimagnetic compounds, such as pure iron, magnetic iron oxide (such as magnetite), gamma-Fe₂O₃, Fe₃O₄, iron sulfides, manganese ferrite, cobalt, ferrite, nickel ferrite, and ferritin filled with magnetite or other magnetically active materials such as ferromagnetic and superparamagnetic materials.

The contrast agents, such as the paramagnetic and superparamagnetic contrast agents described above, can be employed as a component within the vesicles, entrapped within the internal space of the vesicles, administered as a solution with the vesicles or incorporated into the stabilizing compound forming the vesicle wall.

Superparamagnetic agents can be used as clathrates to adsorb and stabilize vesicles. For example, emulsions of various perfluorocarbons, such as perfluorohexane or perfluorochlorocarbons mixed with irregular shaped iron oxide compounds. The hydrophobic clefts in the iron oxides cause nano-droplets of the liquid gaseous precursor to adhere to the surface of the solid material.

For example, if desired, the paramagnetic or superparamagnetic agents can be delivered as alkylated or other derivatives incorporated into the stabilizing compound, especially the lipid walls of the vesicles. In particular, the nitroxides 2,2,5,5-tetramethyl-1-pyrrolidinyloxy, free radical and 2,2,6,6-tetramethyl-1-piperidinyloxy, free radical, can form adducts with long chain fatty acids at the positions of the ring which are not occupied by the methyl groups, via a number of different linkages, e.g., an acetyloxy group. Such adducts are very amenable to incorporation into the stabilizing compounds, especially those of a lipidic nature, which form the walls of the vesicles of the present invention.

Mixtures of any one or more of the paramagnetic agents and superparamagnetic agents in the contrast media may similarly be used.

The paramagnetic and superparamagnetic agents described above may also be coadministered separately, if desired.

The gas-filled vesicles used in the invention methods may not only serve as effective carriers of the superparamagnetic agents, e.g., iron oxides, but also appear to magnify the effect of the susceptibility contrast agents. Superparamagnetic contrast agents include metal oxides, particularly iron oxides but including manganese oxides, and as iron oxides, containing varying amounts of manganese, cobalt and nickel that experience a magnetic domain. These agents are nano or microparticles and have very high bulk susceptibilities and transverse relaxation rates. The larger particles, e.g., 100 nm diameter, have much higher R2 relaxivities than R1 relaxivities, but the smaller particles, e.g., 10 to 15 nm diameter have somewhat lower R2 relaxivities, but much more balanced R1 and R2 values. The smallest particles, e.g., monocrystalline iron oxide particles 3 to 5 nm in diameter, have lower R2 relaxivities, but probably the most balanced R1 and R2 relaxation rates. Ferritin can also be formulated to encapsulate a core of very high relaxation rate superparamagnetic iron. It has been discovered that stabilized gas-filled vesicles used in the present invention can increase the efficacy and safety of these conventional iron oxide based MRI contrast agents.

The iron oxides may simply be incorporated into the stabilizing compounds from which the vesicles are made. Particularly, the iron oxides can be incorporated into the walls of the lipid based vesicles, e.g., adsorbed onto the surfaces of the vesicles, or entrapped within the interior of the vesicles as described in U.S. Pat. No. 5,088,499, issued Feb. 18, 1992.

Although there is no intention to limit the present invention to any particular theory as to its mode of action, it is believed that the vesicles increase the efficacy of the superparamagnetic contrast agents by several mechanisms. First, it is believed that the vesicles function so as to increase the apparent magnetic concentration of the iron oxide particles. Second, it is believed that the vesicles increase the apparent rotational correlation time of the MRI contrast agents, both paramagnetic and superparamagnetic agents, so that relaxation rates are increased. Finally, the vesicles appear to operate by way of a novel mechanism that increases the apparent magnetic domain of the contrast medium and is believed to operate in the manner described immediately below.

The vesicles can be thought of as flexible spherical domains of differing susceptibility from the suspending medium, i.e., the aqueous suspension of the contrast medium and blood in the intravascular space. When considering ferrites or iron oxide particles, it should be noted that these agents have an effect on contrast that depends upon particle size, i.e., it depends on the diameter of the iron oxide particle. This phenomenon is very common and is often referred to as the “secular” relaxation of the water molecules. Described in more physical terms, this relaxation mechanism is dependent upon the effective size of the molecular complex in which a paramagnetic atom, or paramagnetic molecule, or molecules, may reside. One physical explanation can be described by the Solomon-Bloembergen equations, which define the paramagnetic contributions to the T₁ and T₂ relaxation times.

A few large particles will generally have a much greater effect than a larger number of much smaller particles, primarily due to a larger correlation time. If one were to make the iron oxide particles very large however, they might be toxic and embolize the lungs or activate the complement cascade system. Furthermore, it is not the total size of the particle that matters, but particularly the diameter of the particle at its edge or outer surface. The domain of magnetization or susceptibility effect falls off exponentially from the surface of the particle. Generally speaking, in the case of dipolar (through space) relaxation mechanisms, this exponential fall off exhibits an r⁶ dependence. Literally interpreted, a water molecule that is 4 angstroms away from a paramagnetic surface will be influenced 64 times less than a water molecule that is 2 angstroms away from the same paramagnetic surface. The ideal situation in terms of maximizing the contrast effect would be to make the iron oxide particles hollow, flexible and as large as possible. By coating the inner or outer surfaces of the vesicles with the contrast agents, even though the individual contrast agents, e.g., iron oxide nanoparticles or paramagnetic ions, are relatively small structures, the effectiveness of the contrast agents can be greatly enhanced. In so doing, the contrast agents may function as an effectively much larger sphere wherein the effective domain of magnetization is determined by the diameter of the vesicle and is maximal at the surface of the vesicle. These agents afford the advantage of flexibility, i. e., and compliance. While rigid vesicles might lodge in the lungs or other organs and cause toxic reactions, these flexible vesicles slide through the capillaries much more easily.

Methods of Preparation

The stabilized gas-filled vesicles used in the invention methods can be prepared by a number of suitable methods. These are described below separately for the case where the vesicles are gas-filled, and where they are gaseous precursor-filled, although vesicles having both a gas and gaseous precursor are part of the present invention.

Utilizing a Gas

In one example, an aqueous solution comprising a lipid stabilizing compound is agitated in the presence of a gas at a temperature below the gel to liquid crystalline phase transition temperature of the lipid to form vesicles comprising a (i.e., gas-filled vesicles). The term “agitating,” and variations thereof, as used herein, means any motion that shakes an aqueous solution such that gas is introduced from the local ambient environment into the aqueous solution. The shaking must be of sufficient force to result in the formation of vesicles, particularly stabilized vesicles. The shaking can be by swirling, such as by vortexing, side-to-side, or up-and-down motion. Different types of motion can be combined. Also, the shaking may occur by shaking the container holding the aqueous lipid solution, or by shaking the aqueous solution within the container without shaking the container itself.

Further, the shaking may occur manually or by machine. Mechanical shakers that can be used include, for example, a shaker table such as a VWR Scientific (Cerritos, Calif.) shaker table, or a Wig-L-Bug™ Shaker (Crescent Dental Mfg. Ltd., Lyons, Ill.), which has been found to give excellent results. Other shakers that can be used include the Espe Vialmix™ (Bristol Myers-Squibb) or Mixtura™ shaker (ImaRx, Tuscon, Ariz.). Certain modes of shaking or vortexing can be used to make stable vesicles within a preferred size range. For example, shaking carried out using the Wig-L-Bug™ mechanical shaker with a reciprocating motion can be utilized to generate the gas-filled vesicles (e.g., with the motion be reciprocating in the form of an arc such as from about 2° to about 20°, or from about 5° to about 8°, or from about 6° to about 7°, such as about 6.5° can be used. The rate of reciprocation, as well as the arc thereof, is a factor that determines the amount and size of the gas-filled vesicles formed. The number of reciprocations, i.e., full cycle oscillations, can be within the range of from about 1000 to about 20,000 per minute, for example, from about 2500 to about 8000. The Wig-L-Bug™, referred to above, is a mechanical shaker that provides 2000 pestle strikes every 10 seconds, i.e., 6000 oscillations every minute. Of course, the number of oscillations is dependent upon the mass of the contents being agitated, with the larger the mass, the fewer the number of oscillations used. Another means for producing shaking includes the action of gas emitted under high velocity or pressure, for example 3000-4000 RPM.

It will also be understood that, with a larger volume of aqueous solution, the total amount of force will be correspondingly increased. Vigorous shaking is defined as at least about 60 shaking motions per minute. Vortexing at least 60 to about 300, for example 300 to 1800 revolutions per minute can also be used. The formation of gas-filled vesicles upon shaking can be detected visually. The concentration of lipid required to form a desired stabilized vesicle level will vary depending upon the type of lipid used, and can be readily determined by routine experimentation. For example, the concentration of 1,2-dipalimitoyl-phosphatidylcholine (DPPC) used to form stabilized vesicles can be about 0.1 mg/ml to about 30 mg/ml of saline solution, more preferably from about 0.5 mg/ml to about 20 mg/ml of saline solution, for example, from about 1 mg/ml to about 10 mg/ml of saline solution. The concentration of distearoylphosphatidylcholine (DSPC) used can be about 0.1 mg/ml to about 30 mg/ml of saline solution, for example, from about 0.5 mg/ml to about 20 mg/ml of saline solution, or from about 1 mg/ml to about 10 mg/ml of saline solution.

In addition to the simple shaking methods described above, more elaborate methods can also be employed, e.g., liquid crystalline shaking gas instillation processes, and vacuum drying gas instillation processes, such as those described in U.S. Pat. No. 5,580,575, which is incorporated herein by reference, in its entirety. When such processes are used, the stabilized vesicles, which are to be gas-filled, can be prepared prior to gas installation using any one of a variety of conventional liposome preparatory techniques which will be apparent to those skilled in the art. These techniques include freeze-thaw, as well as techniques such as sonication, chelate dialysis, homogenization, solvent infusion, microemulsification, spontaneous formation, solvent vaporization, French pressure cell technique, controlled detergent dialysis, and others, each involving preparing the vesicles in various fashions in a solution containing the desired active ingredient so that the therapeutic, cosmetic or other agent is encapsulated in, enmeshed in, or attached to the resultant polar-lipid based vesicle. See, e.g., Madden et al., Chemistry and Physics of Lipids, (1990) 53:37-46, the disclosure of which is hereby incorporated herein by reference in its entirety.

The gas-filled vesicles prepared in accordance with the methods described above range in size from below a micron to over 12 microns in size. In addition, it will be noted that after the extrusion and sterilization procedures, the agitation or shaking step yields gas-filled vesicles with little to no residual anhydrous lipid phase (Bangham, A. D., Standish, M. M, & Watkins, J. C. (1965) J. Mol. Biol. 13, 238-252) present in the remainder of the solution. The resulting gas-filled vesicles remain stable on storage at room temperature for a year or even longer.

The size of gas-filled vesicles can be adjusted, if desired, by a variety of procedures including microemulsification, vortexing, extrusion, filtration, sonication, homogenization, repeated freezing and thawing cycles, extrusion under pressure through pores of defined size, and similar methods. It may also be desirable to use the vesicles of the present invention as they are formed, without any attempt at further modification of the size thereof.

The sizing or filtration step can be accomplished by the use of a filter assembly when the suspension is removed from a sterile vial prior to use, or even more preferably, the filter assembly can be incorporated into the syringe itself during use. The method of sizing the vesicles will then comprise using a syringe comprising a barrel, at least one filter, and a needle; and will be carried out by a step of extracting which comprises extruding the vesicles from the barrel through the filter fitted to the syringe between the barrel and the needle, thereby sizing the vesicles before they are administered to a patient in the course of using the vesicles in the invention methods as described herein. The step of extracting may also comprise drawing the vesicles into the syringe, where the filter will function in the same way to size the vesicles upon entrance into the syringe. Another alternative is to fill such a syringe with vesicles which have already been sized by some other means, in which case the filter now functions to ensure that only vesicles within the desired size range, or of the desired maximum size, are subsequently administered by extrusion from the syringe.

In preferred embodiments, the stabilizing compound solution or suspension is extruded through a filter and the solution or suspension is heat sterilized prior to shaking. Once gas-filled vesicles are formed, they can be filtered for sizing as described above. These procedures prior to the formation of gas-filled vesicles provide the advantages, for example, of reducing the amount of unhydrated stabilizing compound, and thus providing a significantly higher yield of gas-filled vesicles, as well as and providing sterile gas-filled vesicles ready for administration to a patient. For example, a mixing vessel such as a vial or syringe can be filled with a filtered stabilizing compound, especially lipid suspension, and the suspension can then be sterilized within the mixing vessel, for example, by autoclaving. Gas can be instilled into the lipid suspension to form gas-filled vesicles by shaking the sterile vessel. Preferably, the sterile vessel is equipped with a filter positioned such that the gas-filled vesicles pass through the filter before contacting a patient.

Extruding the stabilizing solution through a filter decreases the amount of unhydrated compound by breaking up the dried compound and exposing a greater surface area for hydration. Preferably, the filter used for this purpose has a pore size of about 0.1 to about 5 microns, for example about 0.1 to about 4 microns or about 0.1 to about 1 or 2 microns Unhydrated compound, especially lipid, appears as amorphous clumps of non-uniform size and is undesirable.

Sterilization provides a composition that can be readily administered to a patient, and can be accomplished by heat sterilization, e.g., by autoclaving the solution at a temperature of at least 100° C. to about 130° C. for at least 1 minute to about 20 minutes, for example, about 15 minutes.

Where sterilization occurs by a process other than heat sterilization to avoid rupture of gas-filled vesicles, sterilization may occur subsequent to the formation of the gas-filled vesicles. For example, gamma radiation can be used before and after gas-filled vesicles are formed.

Utilizing a Gaseous Precursor

In addition to the aforementioned embodiments, one can also use gaseous precursors in the lipid-based vesicles that can, upon activation by temperature, light, or pH, or other properties of the tissues of a host to which it is administered, undergo a phase transition from a liquid or solid entrapped in the lipid-based vesicles, to a gaseous state, expanding to create the stabilized, gas-filled vesicles used in the present invention. This technique is well known in the art and is described in detail in U.S. Pat. Nos. 5,542,935 and 5,585,112, both of which are incorporated herein by reference in their entirety. The techniques for preparing gaseous precursor filled vesicles are generally similar to those described for the preparation of gas-filled vesicles herein, except that a gaseous precursor is substituted for the gas.

The preferred method of activating the gaseous precursor is by temperature. “Activation” or “transition temperature”, and like terms, refer to the boiling point of the gaseous precursor, the temperature at which the liquid to gaseous phase transition of the gaseous precursor takes place. Useful gaseous precursors are those gases that have boiling points in the range of about −100° C. to 70° C. The activation temperature is particular to each gaseous precursor. An activation temperature of about 37° C., or human body temperature, is preferred for gaseous precursors of the present invention. The methods of preparing the vesicles used in the invention methods can be carried out at or below the boiling point of the gaseous precursor, or, for gaseous precursors having low temperature boiling points, liquid precursors can be emulsified using a microfluidizer device chilled to a low temperature. The boiling points may also be depressed using solvents in liquid media to utilize a precursor in liquid form. Further, the methods can be performed where the temperature is increased throughout the process, whereby the process starts with a gaseous precursor as a liquid and ends with a gas.

The gaseous precursor can be selected so as to form the gas in situ in the targeted tissue or fluid, in vivo upon entering the patient or animal, prior to use, during storage, or during manufacture. Activation of the phase transition may take place at any time as the temperature is allowed to exceed the boiling point of the precursor. Also, knowing the amount of liquid in a droplet of liquid gaseous precursor, the size of the vesicles upon attaining the gaseous state can be determined.

Alternatively, the gaseous precursors can be utilized to create stable gas-filled vesicles that are pre-formed prior to use. In this embodiment, the gaseous precursor is added to a container housing a suspending and stabilizing medium at a temperature below the liquid-gaseous phase transition temperature of the respective gaseous precursor. As the temperature is then exceeded, and an emulsion is formed between the gaseous precursor and liquid solution, the gaseous precursor undergoes transition from the liquid to the gaseous state. As a result of this heating and gas formation, the gas displaces the air in the head space above the liquid suspension so as to form gas-filled lipid spheres which entrap the gas of the gaseous precursor, ambient gas (e.g., air) or coentrap gas state gaseous precursor and ambient air. This phase transition can be used for optimal mixing and stabilization of the gas- or gaseous precursor-filled vesicles. For example, the gaseous precursor, perfluorobutane, can be entrapped in the biocompatible lipid or other stabilizing compound, and as the temperature is raised, beyond 4° C. (boiling point of perfluorobutane) stabilizing compound entrapped fluorobutane gas results. As an additional example, the gaseous precursor fluorobutane can be suspended in an aqueous suspension containing emulsifying and stabilizing agents, such as glycerol or propylene glycol, and vortexed on a commercial vortexer. Vortexing is commenced at a temperature low enough that the gaseous precursor is liquid and is continued as the temperature of the sample is raised past the phase transition temperature from the liquid to gaseous state. In so doing, the precursor converts to the gaseous state during the microemulsification process. In the presence of the appropriate stabilizing agents, surprisingly stable gas-filled vesicles result.

Accordingly, the gaseous precursors can be selected to form a gas-filled vesicle in vivo or can be designed to produce the gas-filled vesicle in situ, during the manufacturing process, on storage, or at some time prior to use.

As a further embodiment of this invention, by pre-forming the liquid state of the gaseous precursor into an aqueous emulsion and maintaining a known size, the maximum size of the microbubble can be estimated by using the ideal gas law,

Taking advantage of principles in the ideal gas law and the expansion in size of the vesicles from the liquid to gaseous phases stable vesicles that are small enough to be injected through in line filters and provide the necessary contrast enhancement in vivo can be made. Indeed, knowing the expansion in microsphere diameter upon liquid to gaseous transition a filter system may be designed such that the particles or emulsion is sized via a process of injection/filtration. Upon transition from the liquid to gaseous phases, the appropriate sized gas-filled vesicles will the form. Knowing the necessary volume of gaseous precursor and the contribution of the stabilizing materials to effective droplet diameter and then utilizing the ideal gas law, the optimal filter diameter for sizing the precursor droplets may be calculated. This, in turn, will produce vesicles of the desired diameter. The gaseous precursor-filled vesicles may also be sized by a simple process of extrusion through filters.

This embodiment for preparing gas-filled vesicles used in the invention methods can be applied to all gaseous precursors activated by temperature. In fact, depression of the freezing point of the solvent system allows the use gaseous precursors that would undergo liquid-to-gas phase transitions at temperatures below 0° C. The solvent system can be selected to provide a medium for suspension of the gaseous precursor. For example, 20% propylene glycol miscible in buffered saline exhibits a freezing point depression well below the freezing point of water alone. By increasing the amount of propylene glycol or adding materials such as sodium chloride, the freezing point can be depressed even further. The selection of appropriate solvent systems can be explained by physical methods as well. When substances, solid or liquid, herein referred to as solutes, are dissolved in a solvent, such as water-based buffers for example, the freezing point is lowered by an amount that is dependent upon the composition of the solution. Thus, as defined by Wall, one can express the freezing point depression of the solvent by the following equation:

lnxₐ=ln(1−x_{b})=ΔH_{fus}/R(1/T₀−1/T)

where: xₐ=mole fraction of the solvent; x_{b}=mole fraction of the solute; ΔH_{fus}=heat of fusion of the solvent; and T₀=Normal freezing point of the solvent.

The normal freezing point of the solvent results from solving the equation. The above equation can be used to accurately determine the molal freezing point of gaseous-precursor filled vesicle solutions used in the present invention. Hence, the above equation can be applied to estimate freezing point depressions and to determine the appropriate concentrations of liquid or solid solute necessary to depress the solvent freezing temperature to an appropriate value.

Methods of preparing the temperature activated gaseous precursor-filled vesicles include:

(a) vortexing an aqueous suspension of gaseous precursor-filled vesicles used in the present invention; variations on this method include optionally autoclaving before shaking, optionally heating an aqueous suspension of gaseous precursor and lipid, optionally venting the vessel containing the suspension, optionally shaking or permitting the gaseous precursor vesicles to form spontaneously and cooling down the gaseous precursor filled vesicle suspension, and optionally extruding an aqueous suspension of gaseous precursor and lipid through a filter of about 0.22 micron, alternatively, filtering can be performed during in vivo administration of the resulting vesicles such that a filter of about 0.22 micron is employed;

(b) a microemulsification method whereby an aqueous suspension of gaseous precursor-filled vesicles of the present invention are emulsified by agitation and heated to form vesicles prior to administration to a patient; and

(c) forming a gaseous precursor in lipid suspension by heating, and agitation, whereby the less dense gaseous precursor-filled vesicles float to the top of the solution by expanding and displacing other vesicles in the vessel and venting the vessel to release air; and (d) in any of the above methods, utilizing a sealed vessel to hold the aqueous suspension of gaseous precursor and stabilizing compound such as biocompatible lipid, the suspension being maintained at a temperature below the phase transition temperature of the gaseous precursor, followed by autoclaving to move the temperature above the phase transition temperature, optionally with shaking, or permitting the gaseous precursor vesicles to form spontaneously, whereby the expanded gaseous precursor in the sealed vessel increases the pressure in the vessel, and cools the gas-filled vesicle suspension.

Freeze drying is useful to remove water and organic materials from the stabilizing compounds prior to the shaking gas instillation method. Drying-gas instillation methods can be used to remove water from vesicles. By pre-entrapping the gaseous precursor in the dried vesicles (i.e., prior to drying) after warming, the gaseous precursor may expand to fill the vesicle. Gaseous precursors can also be used to fill dried vesicles after they have been subjected to vacuum. As the dried vesicles are kept at a temperature below their gel/liquid crystalline transition temperature, the drying chamber can be slowly filled with the gaseous precursor in its gaseous state, e.g., perfluorobutane can be used to fill dried vesicles composed of dipalmitoylphosphatidylcholine (DPPC) at temperatures between 4° C. (the boiling point of perfluorobutane) and below 40° C., the phase transition temperature of the biocompatible lipid. In this case, the vesicles could be filled at a temperature of about 4° C. to about 5° C.

Preferred methods for preparing the temperature activated gaseous precursor-filled vesicles comprise shaking an aqueous solution having a stabilizing lipid compound, such as a biocompatible lipid, in the presence of a gaseous precursor at a temperature below the gel state to liquid crystalline state phase transition temperature of the lipid or shaking an aqueous solution comprising a stabilizing compound such as a biocompatible lipid in the presence of a gaseous precursor, and separating the resulting gaseous precursor-filled vesicles. Vesicles prepared by the foregoing methods are referred to herein as gaseous precursor-filled vesicles prepared by a gel state shaking gaseous precursor instillation method.

Conventional, aqueous-filled liposomes of the prior art are routinely formed at a temperature above the phase transition temperature of the lipids used to make them, since they are more flexible and thus useful in biological systems in the liquid crystalline state. See, for example, Szoka and Papahadjopoulos, Proc. Natl. Acad. Sci. (1978), 75d:4194-4198. In contrast, the gaseous precursor-filled vesicles have greater flexibility, since gaseous precursors after gas formation are more compressible and compliant than an aqueous solution. Thus, the gaseous precursor-filled vesicles can be utilized in biological systems when formed at a temperature below the phase transition temperature of the lipid, even though the gel phase is more rigid.

The methods contemplated by the present invention provide for shaking an aqueous solution comprising a stabilizing compound such as a biocompatible lipid in the presence of a temperature activated gaseous precursor. Shaking, as used herein, is defined as a motion that agitates an aqueous solution such that gaseous precursor is introduced from the local ambient environment into the aqueous solution. Any type of motion that agitates the aqueous solution and results in the introduction of gaseous precursor can be used for the shaking. The shaking must be of sufficient force to allow the formation of a suitable number of vesicles after a period of time. Preferably, the shaking is of sufficient force such that vesicles are formed within a short period of time, such as about 10 to 30 minutes. The shaking can be by microemulsifying, by microfluidizing, for example, with a swirling (such as by vortexing), side-to-side, or up and down motion. In the case of the addition of gaseous precursor in the liquid state, sonication can be used in addition to the shaking methods set forth above. Further, different types of motion can be combined. Also, the shaking may occur by shaking the container holding the aqueous lipid solution, or by shaking the aqueous solution within the container without shaking the container itself. Further, the shaking may occur manually or by machine. Mechanical shakers that can be used include, for example, a shaker table, such as a VWR Scientific (Cerritos, Calif.) shaker table, a microfluidizer, Wig-L-Bug™ (Crescent Dental Manufacturing, Inc., Lyons, Ill.), which has been found to give particularly good results, and a mechanical paint mixer, as well as other known machines. Another means for producing shaking includes the action of gaseous precursor emitted under high velocity or pressure. It will also be understood that with a larger volume of aqueous solution, the total amount of force will be correspondingly increased. Vigorous shaking is defined as at least about 60 shaking motions per minute. Vortexing at least 1000 to 1800 revolutions per minute, are examples of vigorous shaking.

The formation of gaseous precursor-filled vesicles upon shaking can be detected by the presence of foam on the top of the aqueous solution coupled with a decrease in the volume of the aqueous solution. The final volume of the foam is generally at least about two times the initial volume of the aqueous lipid solution and under some conditions all of the aqueous lipid solution is converted to foam.

The required duration of shaking time can be determined by detection of the formation of foam. For example, 10 ml of lipid solution in a 50 ml centrifuge tube can be vortexed for approximately 15-20 minutes or until the viscosity of the gaseous precursor-filled vesicles becomes sufficiently thick so that it no longer clings to the sidewalls as it is swirled. At this time, the foam may cause the solution containing the gaseous precursor-filled vesicles to rise to a level of 30 to 35 ml.

The concentration of lipid stabilizing compound required to form a suitable foam level will vary depending upon the type of stabilizing biocompatible lipid used, and can be readily determined by one skilled in the art, once armed with the present disclosure. For example, the concentration of 1,2-dipalmitoylphosphatidylcholine (DPPC) used to form gaseous precursor-filled vesicles according to methods contemplated by the present invention is about 20 mg/ml to about 30 mg/ml saline solution while the concentration of distearoylphosphatidylcholine (DSPC) used is about 5 mg/ml to about 10 mg/ml saline solution. Specifically, DPPC in a concentration of 20 mg/ml to 30 mg/ml, upon shaking, yields a total suspension and entrapped gaseous precursor volume four times greater than the suspension volume alone. DSPC in a concentration of 10 mg/ml, upon shaking, yields a total volume completely devoid of any liquid suspension volume and contains entirely foam.

It will be understood by one skilled in the art, once armed with the present disclosure, that the lipids and other stabilizing compounds used as starting materials, or the vesicle final products, can be manipulated prior and subsequent to being subjected to the methods described herein. For example, the stabilizing biocompatible lipid can be hydrated and then lyophilized, processed through freeze and thaw cycles, or simply hydrated. Alternatively, the lipid is hydrated and then lyophilized, or hydrated, then processed through freeze and thaw cycles and then lyophilized, prior to the formation of gaseous precursor-filled vesicles.

A gas can be injected into or otherwise added to the container having the aqueous lipid solution or into the aqueous lipid solution itself in order to provide a gas other than air. Gases that are not heavier than air can be added to a sealed container while gases heavier than air can be added to a sealed or an unsealed container. Accordingly, the present invention includes co-entrapment of air and other gases along with gaseous precursors.

As already described above in the section dealing with the stabilizing compound, the preferred methods contemplated by the present invention are carried out at a temperature below the gel/liquid crystalline transition temperature of the lipid employed. Hence, the stabilized vesicle precursors described above, can be used in the same manner as the other stabilized vesicles used in the present invention, once activated by application to the tissues of a host, where such factors as body temperature or pH can be used to cause generation of the gas. Where the host tissue is human tissue having a normal temperature of about 37° C., the gaseous precursors advantageously undergo phase transitions from liquid to gaseous states near 37° C.

All of the above embodiments involving preparations of the stabilized gas-filled vesicles used in the invention methods , can be sterilized by autoclave or sterile filtration if these processes are performed before either the gas instillation step or prior to temperature mediated gas conversion of the temperature sensitive gaseous precursors within the suspension. Alternatively, one or more biocompatible anti-bactericidal agents and preservatives can be included in the formulation of the vesicles. Such sterilization, which may also be achieved by other conventional means, such as by irradiation, will be necessary because the stabilized vesicles are used for intravascular administration. The appropriate means of sterilization will be apparent to those of skill in the art instructed by the present description of the stabilized gas-filled vesicles and their use. The vesicles are generally stored as an aqueous suspension but in the case of dried vesicles or dried lipidic spheres can be stored as a dried powder ready to be reconstituted prior to use.

The invention is further demonstrated in the following examples, which are intended to illustrate, but not in any way to limit the scope of the present invention.

### EXAMPLES

### Example 1

A. Perflutren lipid microspheres used extensively in clinical imaging at MI=0.8 have shown no evidence of any local tissue damage due to application of ultrasound. Therefore, ultrasound energy levels were selected for testing such that the mechanical index (MI) was less than 0.8. The ultrasound energy level was also selected to minimize heat generated and hence discomfort experienced by a person upon application of ultrasound. At the vesicle size used (ranging from about 1 to 2 microns), 1.0 MHz was selected as being close to the peak resonant frequency for the bubbles.

To test the effectiveness of various ultrasound parameters for lysing blood clots, in vitro experiments were performed for samples at 1.0 MHz and different power intensities ranging from 0.75 Watts/cm² (100% duty cycle) to 10.0 Watts/cm² (10% duty cycle). Some of the successful parameters were found to be:

a) 1 MHz, 0.75 Watts/cm²,100% duty cycle

b) 1 MHz, 0.75 Watts/cm²,10% duty cycle

c) 1 MHz, 1.5 Watts/cm²,100% duty cycle

d) 1 MHz, 2.0 Watts/cm²,10% duty cycle

e) 1 MHz, 2.0 Watts/cm²,20% duty cycle

f) 1 MHz, 10 Watts/cm²,10% duty cycle

B. Blood from healthy human volunteers was doped with trace amounts of fibrinogen labeled with a fluorescent probe. The extent of clot lysis was measured by the increase in fluorescence of the plasma overlay resulting from release of fluorescently labeled fibrinogen upon clot lysis.

Blood clots were formed by modification of the procedure described in Suchkova, et al. (Circulation (1998) 98:1030-1035). Briefly, each clot was formed on a thread in a plastic tube (Beckman) by incubating 160 μL of blood doped with 10 μg of Alexafluor®-594 labeled fibrinogen (F-13193 Molecular Probes, OR), 8 uL of 1 × thrombin (prepared from 100× thrombin; Sigma Chemicals) and 3.2 μL of 1M CaCl₂ (Fluka) at 37° C. for one hour. Blood clots were suspended in 820 μL of heparinized plasma (U.S. Biological, Swampscott, Mass.). The clot lysis experiments with ultrasound were carried out using a 1 MHz Rich-Mar AutoSound Model No. 5.6 device equipped with a 5 cm² probe at a power level of 2 W/cm² at a 10% duty cycle (Rich-Mar, Inola, Okla.), following which the unlysed clot was discarded and the plasma solution spun down to pellet the residual red blood cells. Fluorescence of the supernatant plasma was measured in a 96-well plate using an F-Max plate reader (Molecular Devices, Sunnyvale, Calif.). The excitation wavelength was 584 nm and the emission wavelength was 612 nm. The data reported is an average of six trials of the following experiments (n=6).

100 ng tPA added to the overlay

100 ng tPA and 2 μL of MRX-133 added to the overlay. Three such additions were made every 20 minutes for a total of 6 uL of MRX-133

Two ultrasound experimental controls were done simultaneously:

2 μL of MRX-133 was added three times in the absence of tPA

No MRX-133 was added three times in the absence of tPA

Two control experiments were also done without ultrasound:

100 ng of tPA was added to the plasma

No MRX-133 or tPA was added to the plasma

A table outlining the experimental design is presented below:

TABLE 3

| | Frequency/ | | |
|---|---|---|---|
| Experiment | Power/Duty cycle | MRX-133 | tPA |
| No ultrasound control | N/A | N/A | N/A |
| Ultrasound control | 1 MHz/2 Watts/cm²/10% | N/A | N/A |
| Ultrasound + MRX-133 | 1 MHz/2 Watts/cm²/10% | 3 × 2 μL | N/A |
| tPA Control | N/A | N/A | 100 ng |
| tPA + ultrasound | 1 MHz/2 Watts/cm²/10% | N/A | 100 ng |
| tPA + ultrasound + | 1 MHz/2 Watts/cm²/10% | 3 × 2 μL | 100 ng |
| MRX-133 | | | |

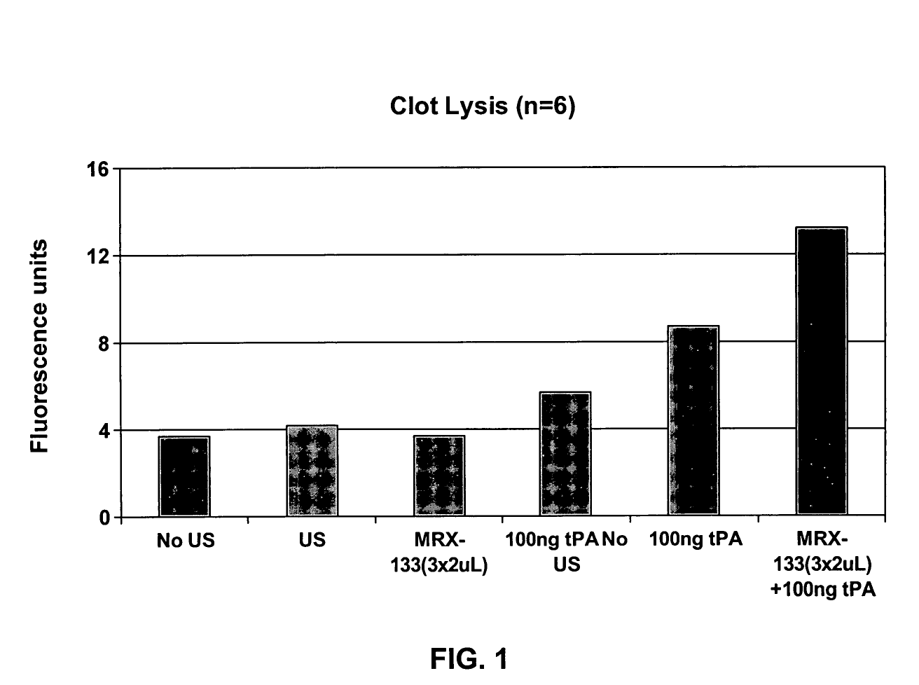

A striking 2.4-fold increase in clot lysis was observed upon insonation of blood clots in the presence of 6 μL of MRX-133 gas-filled vesicles compared to tPA alone using no ultrasound ore gas-filled vesicles (FIG. 1). Compared to tPA plus ultrasound, the addition of MRX-133 gas-filled vesicles resulted in more than 50% clot lysis. All samples contained 100 ng of tPA, which approximates the level of free tPA found in serosal fluids during surgery.

The disclosures of each patent, patent application and publication cited or described in this document are hereby incorporated herein by reference in their entirety.

Various modifications of the invention in addition to those described herein will be apparent to those of skill in the art from the foregoing description. Such modifications are also intended to fall within the scope of the appended claims.

What is claimed is:

1. A non-invasive method for disrupting a blood clot within the vasculature of a patient, the method comprising:
(a) administering intravascularly to the patient a vesicle composition comprising, in an aqueous carrier, a lipid and a gas or gaseous precursor; and
(b) applying to the patient ultrasound having a power greater than about 0.5 Watts/cm² to about 20 Watts/cm² for about 10% to about 80% of the duty cycle for a period of time sufficient to induce rupture of the vesicles adjacent to the site of the blood clot, thereby disrupting the blood clot.

2. The method of claim 1, further comprising scanning the patient with diagnostic imaging to monitor disruption of the blood clot.

3. The method of claim 2, wherein the scanning is performed prior to, simultaneously with, or after application of the ultrasound.

4. The method of claim 3, wherein the diagnostic imaging comprises magnetic resonance imaging (MRI).

5. The method of claim 1, wherein the period of time is about 1 minute to about 8 hours.

6. The method of claim 5, wherein the period of time is about 5 minutes to about 2 hours.

7. The method of claim 6, wherein the period of time is for about 1 hour.

8. The method of claim 1, wherein the ultrasound is focused.

9. The method of claim 1, wherein the ultrasound is non-focused.

10. The method of claim 1, wherein mechanical index of the ultrasound is no greater than about 8.0.

11. The method of claim 1, wherein the power is 10 Watts/cm² delivered at 50% of the duty cycle.

12. The method of claim 1, wherein the ultrasound is delivered at from about 0.1% to less than 80% of the duty cycle.

13. The method of claim 1, wherein the blood clot is in the vasculature of the brain.

14. The method of claim 1, wherein the blood clot is associated with rupture of a vulnerable plaque in the vasculature.

15. The method of claim 1, wherein the blood clot is associated with ischemic or hemorrhagic stroke.

16. The method of claim 1, wherein the blood clot is associated with an atherosclerotic plaque.

17. The method of claim 1, wherein the blood clot results from an interventional medical procedure.

18. The method of claim 1, wherein the blood clot results from acute limb ischemia.

19. The method of claim 1, wherein the blood clot is associated with a myocardial infarction.

20. The method of claim 1, wherein the blood clot is associated with a dialysis graft.

21. The method of claim 1, wherein the blood clot is associated with deep vein thrombosis.

22. The method of claim 1, wherein the administration is intravenously.

23. The method of claim 1, wherein the administration is intraarterially.

24. The method of claim 1, wherein the vesicles further comprise a targeting ligand.

25. The method of claim 24, wherein the blood clot is in a vein and the targeting ligand targets fibrin.

26. The method of claim 24, wherein the blood clot is in an artery and the targeting ligand targets platelets.

27. The method of claim 1 wherein the vesicles further comprise a therapeutic agent that is released upon application of the ultrasound.

28. The method of claim 27, wherein the therapeutic agent is a thrombolytic.

29. The method of claim 27, wherein the therapeutic agent is tissue plasminogen activator (tPA).

30. The method of claim 1, wherein the composition further comprises a drug.

31. The method of claim 1, wherein the composition further comprises an anti-coagulant.

32. The method of claim 31, wherein the anti-coagulant is a heparin.

33. The method of claim 1, wherein the method further comprises co-administration of a antihyperlipidemic agent.

34. The method of claim 1 wherein the gas or gaseous precursor are perfluorocarbons containing less than 10 carbon atoms.

35. The method of claim 34, the perfluorocarbons are selected from the group consisting of perfluoropropane, perfluorobutane, perfluorocyclobutane, perfluoromethane, perfluoroethane, perfluorohexane, and perfluoropentane.

36. The method of claim 34, wherein the perfluorocarbon compound is perfluoropropane.

37. The method of claim 34, wherein the perfluorocarbon compound is perfluorobutane.

38. The method of claim 1, wherein the vesicles comprise liposomes.

39. The method of claim 4, wherein the composition further comprises a paramagnetic agent.

40. The method of claim 39, wherein the paramagnetic agent comprises a paramagnetic ion selected from the group consisting of transition, lanthanide and actinide elements.

41. The method of claim 4, wherein the vesicles have an average diameter of about 1 to about 5 microns.

42. The method of claim 41, wherein the vesicles have an average diameter of about 1 to about 3 microns.
